(19) United States
(12) Patent Application Publication
(10) Pub. No.: US 2020/0402227 A1
(43) Pub. Date: Dec. 24, 2020

(54) IMAGE PROCESSING APPARATUS, AND IMAGE PROCESSING METHOD, AND PROGRAM

(71) Applicant: SONY CORPORATION, TOKYO (JP)

(72) Inventors: SHINICHIRO GOMI, TOKYO (JP); ATSUSHI ITO, KANAGAWA (JP); TAKESHI UEMORI, TOKYO (JP)

(21) Appl. No.: 16/769,969

(22) PCT Filed: Nov. 30, 2018
(86) PCT No.: PCT/JP2018/044129

§ 371 Date: Jun. 4, 2020

(30) Foreign Application Priority Data
Dec. 15, 2017 (JP) 2017-240526

(51) Int. Cl. G06T 7/00; G01B 11/30; G01J 3/28

(52) U.S. Cl. CPC G06T 7/0012; G06T 2207/30088; G01J 3/2823; G01B 11/303

(57) ABSTRACT

The present disclosure relates to an image processing apparatus having a simple configuration and enabled to evaluate a state of a subject in view of an adverse effect of an ambient light source, and an image processing method, and a program. A difference square root sum between a subject spectral characteristic corresponding to a spectral characteristic of a skin of a user used as a subject in a captured image and a spectral characteristic of a preferred state is determined, and on the basis of comparison of the difference square root sum with a predetermined threshold, the state of the skin, used as a subject, is evaluated, and a recommended product corresponding to the evaluation result is presented. The present disclosure can be applied to a recommended product presenting apparatus.

### TECHNICAL FIELD

The present disclosure relates to an image processing apparatus, and an image processing method, and a program, and in particular to an image processing apparatus having a simple configuration and enabled to evaluate the state of a subject taking an adverse effect of an ambient light source into account, and an image processing method, and a program.

### BACKGROUND ART

Techniques have been proposed in which an image of a human skin is captured as a subject and in which, on the basis of the captured image of the skin, the state of the skin is measured and evaluated.

For example, techniques have been proposed in which an image of the skin is captured and in which, on the basis of the captured image, a vascular network in the skin or the state of pores in the skin, or the color of a cosmetic ingredient applied to the skin are diagnosed (see PTL 1 and PTL 2).

### CITATION LIST

### Patent Literature

### [PTL 1]

JP 2002-263084 A

### [PTL 2]

JP 2002-189918 A

### SUMMARY

### Technical Problem

However, in the techniques in both PTL 1 and PTL 2, the state of the skin is measured, for example, with the adverse effect of an ambient light source eliminated by, for example, making no consideration for the adverse effect of the ambient light source during measurement, using a dedicated skin measuring apparatus, or capturing an image of a reference color chart simultaneously with capturing of an image of the skin.

Thus, in a case where an image of the skin, used as a subject, is captured and the skin color is measured on the basis of an image capturing result, color information related to the skin as a subject fails to be simply and accurately acquired, adjustment of the ambient light source or simultaneous capturing of an image of the reference color chart is required. This is burdensome in accurately evaluating the state of the subject.

In light of these circumstances, an object of the present disclosure is particularly to allow the state of the subject to be accurately evaluated by using a simple configuration and taking the adverse effect of the ambient light source into account.

### Solution to Problem

An image processing apparatus according to an aspect of the present disclosure is an image processing apparatus including an evaluation section evaluating a state of a subject in a captured image on the basis of a subject spectral characteristic corresponding to a spectral characteristic of the subject and a reference spectral characteristic.

The evaluation section can be caused to evaluate the state of the subject on the basis of a difference between the subject spectral characteristic and the reference spectral characteristic.

The evaluation section can be caused to output, as an evaluation result for the state of the subject, a comparison result of comparison of a difference square root sum between the subject spectral characteristic and the reference spectral characteristic with a predetermined threshold.

The predetermined threshold can be a difference square root sum between an average value of subject spectral characteristics of a plurality of subjects and the reference spectral characteristic.

The reference spectral characteristic can be a spectral characteristic of a preferred state of the subject in the subject spectral characteristic.

An identification section can further be provided that identifies an article with a spectral characteristic applied to the subject to make the spectral characteristic of the subject more similar to the reference spectral characteristic, and a display section can further be provided that displays the article identified by the identification section.

The identification section can be caused to identify, on the basis of the difference between the subject spectral characteristic and the reference spectral characteristic, an article with a spectral characteristic applied to the subject to make the spectral characteristic of the subject more similar to the reference spectral characteristic, in a case where, in an evaluation result for the state of the subject including a comparison result of comparison of a difference square root sum between the subject spectral characteristic and the reference spectral characteristic with the predetermined threshold, the difference square root sum between the subject spectral characteristic and the reference spectral characteristic is larger than the predetermined threshold.

The spectral characteristic applied to the subject to make the spectral characteristic of the subject more similar to the reference spectral characteristic may be a spectral characteristic obtained by multiplying, for each wavelength, the difference between the subject spectral characteristic and the reference spectral characteristic by a predetermined coefficient and adding the subject spectral characteristic to a result of the multiplication.

An article storage section can further be provided that stores information related to the article in association with information related to a spectral characteristic provided in the subject by application of the article, and the identification section can be caused to identify an article included in articles stored in the article storage section, the article involving a decrease in a difference square root sum between the spectral characteristic stored in association with the information and a spectral characteristic applied to the subject to make the spectral characteristic of the subject more similar to the reference spectral characteristic.

The evaluation section can be caused to evaluate the state of the subject by classifying the subject spectral characteristic.

The evaluation section can be caused to evaluate the state of the subject by dividing the subject spectral characteristic into a plurality of wavelength regions, classifying the subject spectral characteristic on the basis of a comparison result of comparison of a predetermined threshold with a difference square root sum for each wavelength region resulting from the division, and obtaining a classification result as an evaluation index.

An identification section can be further provided that identifies, on the basis of the evaluation index, an article with a spectral characteristic applied to the subject to make the spectral characteristic of the subject more similar to the reference spectral characteristic, and a display section can be further provided that displays the article identified by the identification section.

An article storage section can further be provided that stores, for information related to the article, an index of the article with a spectral characteristic applied to the subject to make the spectral characteristic of the subject more similar to the reference spectral characteristic, in association with an evaluation index of the subject spectral characteristic, the index of the article being based on a difference between the subject spectral characteristic and the reference spectral characteristic, and the identification section can be caused to identify an article that is included in articles stored in the article storage section and that is stored in association with the evaluation index, as an article with a spectral characteristic applied to the subject to make the spectral characteristic of the subject more similar to the reference spectral characteristic.

The spectral characteristic applied to the subject to make the spectral characteristic of the subject more similar to the reference spectral characteristic is a spectral characteristic obtained by multiplying, for each wavelength, the difference between the subject spectral characteristic and the reference spectral characteristic by a predetermined coefficient and adding the subject spectral characteristic to a result of the multiplication.

An ambient-light-source spectral characteristic estimating section can further be provided that estimates a spectral characteristic of an ambient light source in a captured image as an ambient-light-source spectral characteristic, and the evaluation section can be caused to evaluate a state of the subject in an image with an adverse effect of the ambient light source in the image reduced using the ambient-light-source spectral characteristic estimated from the image, on the basis of a difference between the reference spectral characteristic and the subject spectral characteristic of the subject

An ambient-light-source spectral characteristic storage section can further be provided that stores, in association with a measurement location, the ambient-light-source spectral characteristic estimated by the ambient-light-source spectral characteristic estimating section, and the evaluation section can be caused to evaluate, on the basis of the subject spectral characteristic of the subject, the state of the subject in an image with the adverse effect of the ambient light source in the image reduced using an ambient-light-source spectral characteristic selected from ambient-light-source spectral characteristics stored in the ambient-light-source spectral characteristic storage section.

An inappropriate-ambient-light-source detecting section can further be provided that detects that the ambient light source is an inappropriate light source for the subject spectral characteristic on the basis of the ambient-light-source spectral characteristic estimated by the ambient-light-source spectral characteristic estimating section, and a presentation section can further be provided that indicates that the ambient light source is an inappropriate light source in a case where the ambient light source is detected as an inappropriate light source.

The inappropriate-ambient-light-source detecting section can be caused to detect that the light source is inappropriate on the basis of a comparison between an average value of the subject spectral characteristic in the ambient-light-source spectral characteristic estimated by the ambient-light-source spectral characteristic estimating section.

An image processing method according to an aspect of the present disclosure is an image processing method including evaluation processing of evaluating a state of a subject in a captured image on the basis of a reference spectral characteristic and a subject spectral characteristic corresponding to a spectral characteristic of the subject.

A program according to an aspect of the present disclosure is a program causing an evaluation section evaluating a state of a subject in a captured image to function as a computer on the basis of a reference spectral characteristic and a subject spectral characteristic corresponding to a spectral characteristic of the subject.

In an aspect of the present disclosure, the state of the subject in the captured image is evaluated on the basis of the reference spectral characteristic and the subject spectral characteristic corresponding to the spectral characteristic of the subject.

### Advantageous Effect of Invention

According to an aspect of the present disclosure, in particular, a skin state can be accurately evaluated by using a simple configuration and taking the adverse effect of the ambient light source into account.

### BRIEF DESCRIPTION OF DRAWINGS

FIG. 1 is a block diagram illustrating a configuration example of a first embodiment of a recommended product presenting apparatus according to the present disclosure.

FIG. 2 is a diagram illustrating a preferred subject spectral characteristic to be compared when an estimated subject spectral characteristic is evaluated.

FIG. 3 is a diagram illustrating examples of data related to the preferred subject spectral characteristic stored in a characteristic classification storage section.

FIG. 4 is a diagram illustrating examples of data related to an average subject spectral characteristic stored in the characteristic classification storage section.

FIG. 5 is a diagram illustrating a suitable subject spectral characteristic determined from the estimated subject spectral characteristic and the preferred subject spectral characteristic.

FIG. 6 is a diagram illustrating examples of product information stored in association with the subject spectral characteristic of a preferred state stored in a product storage section.

FIG. 7 is a diagram illustrating an ambient-light-source spectral characteristic appropriate for the subject spectral characteristic and an ambient-light-source spectral characteristic inappropriate for the subject spectral characteristic.

FIG. 8 is a diagram illustrating an example of display of a recommended product.

FIG. 9 is a diagram illustrating examples of warning indicating that an ambient light source is inappropriate.

FIG. 10 is a flowchart illustrating product recommendation processing executed by the recommended product presenting apparatus in FIG. 1.

FIG. 11 is a block diagram illustrating a configuration example of a second embodiment of the recommended product presenting apparatus according to the present disclosure.

FIG. 12 is a diagram illustrating an example in which an estimated subject spectral characteristic is classified.

FIG. 13 is a flowchart illustrating product recommendation processing executed by the recommended product presenting apparatus in FIG. 11.

FIG. 14 is a block diagram illustrating a configuration example of a third embodiment of the recommended product presenting apparatus according to the present disclosure.

FIG. 15 is a diagram illustrating a wavelength region used for each measurement item when evaluation is performed using the subject spectral characteristic.

FIG. 16 is a flowchart illustrating product recommendation processing executed by the recommended product presenting apparatus in FIG. 14.

FIG. 17 is a diagram illustrating an example of display of a selection screen for the measurement item.

FIG. 18 is a block diagram illustrating a configuration example of a fourth embodiment of the recommended product presenting apparatus according to the present disclosure.

FIG. 19 is a flowchart illustrating product recommendation processing executed by the recommended product presenting apparatus in FIG. 18.

FIG. 20 is a diagram illustrating an example of display of a measurement environment selection screen for selection of an ambient-light-source spectral characteristic.

FIG. 21 is a block diagram illustrating a configuration example of a fifth embodiment of the recommended product presenting apparatus according to the present disclosure.

FIG. 22 is a flowchart illustrating product recommendation processing executed by the recommended product presenting apparatus in FIG. 21.

FIG. 23 is a diagram illustrating a configuration example of a general-purpose computer.

### DESCRIPTION OF EMBODIMENTS

Suitable embodiments of the present disclosure will be described below in detail with reference to the accompanying drawings. Note that, in the present specification and the drawings, components with substantially the same functions and configurations are denoted by the same reference numerals, with duplicate descriptions omitted.

Embodiments of the present technique will be described below. The descriptions are made in the following order.

1. First Embodiment

2. Second Embodiment

3. Third Embodiment

4. Fourth Embodiment

5. Fifth Embodiment

6. Example of Execution by Using Software

### 1. First Embodiment

The present disclosure is configured to accurately estimate a spectral characteristic of skin by using a simple configuration and taking the adverse effect of an ambient light source into account and to evaluate the state of the skin on the basis of the estimation result.

More specifically, a recommended product presenting apparatus (image processing apparatus) according to the present disclosure estimates the spectral characteristic of the skin of the user by using the simple configuration and taking the adverse effect of the ambient light source into account, and comparing the spectral characteristic with a reference spectral characteristic used as a preferred spectral characteristic to evaluate the state of the skin of the user. The recommended product presenting apparatus according to the present disclosure selects (identifies) and presents, on the basis of the evaluation result, a recommended product (recommend product or article) that applies processing of providing the skin with a suitable spectral characteristic.

Note that, in the present disclosure, the evaluation of the state of the skin of the user is evaluation for a comparison between the spectral characteristic of the skin, used as a subject for which an image has been captured, and a preferred spectral characteristic of a preferred state of the skin. Additionally, the comparison between the spectral characteristics relates to an average value, a maximum value, and a minimum value for an entire wavelength region, a predetermined wavelength region, or each of division wavelength regions into which the entire wavelength region is divided, and includes a simple comparison of values between the spectral characteristics, a difference between the spectral characteristics itself or a comparison between the difference and a predetermined threshold, a comparison between a predetermined threshold and a ratio between the spectral characteristics, and a combination thereof.

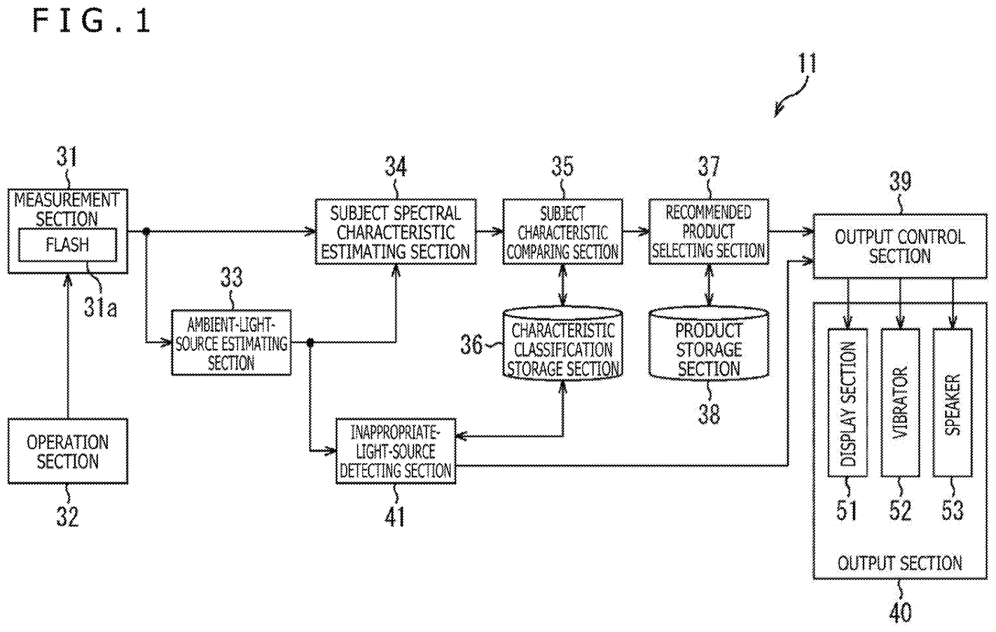

With reference to a block diagram in FIG. 1, a configuration example that implements functions of the recommended product presenting apparatus according to the present disclosure will be described below.

A recommended product presenting apparatus 11 in FIG. 1 includes, for example, a portable terminal represented by a smartphone. The recommended product presenting apparatus 11 includes a measurement section 31, an operation section 32, an ambient-light-source estimating section 33, and a subject spectral characteristic estimating section 34. Furthermore, the recommended product presenting apparatus 11 includes a subject characteristic comparing section 35, a characteristic classification storage section 36, a recommended product selecting section 37, a product storage section 38, an output control section 39, an output section 40, and an inappropriate-light-source detecting section 41.

The measurement section 31 includes a CMOS (Complementary Metal Oxide Semiconductor) image sensor, measures the skin of a user used as a subject by capturing an image of the skin, and outputs the captured image to the ambient-light-source estimating section 33 and the subject spectral characteristic estimating section 34. Additionally, the measurement section 31 includes a flash 31a, and during image capturing, consecutively captures an image with the flash 31a on and an image with the flash 31a off to capture a total of two images and outputs the images.

The operation section 32 includes operation buttons and a keyboard, and is operated by the user at a timing when the measurement section 31 captures images to output, to the measurement section 31, an operation signal corresponding to the operation content.

The ambient-light-source estimating section 33 estimates the spectral characteristic of the ambient light source during image capturing on the basis of an image of the skin of the user corresponding to the measurement result fed by the measurement section 31, and outputs the spectral characteristic to the subject spectral characteristic estimating section 34 and the inappropriate-light-source detecting section 41 as an ambient-light-source spectral characteristic.

On the basis of the image of the skin of the user corresponding to the measurement result fed by the measurement section 31, the subject spectral characteristic estimating section 34 uses the ambient-light-source spectral characteristic to eliminate (reduce) the adverse effect of the ambient light source and estimates the spectral characteristic of the subject during image capturing and outputs the estimated spectral characteristic to the subject characteristic comparing section 35 as a subject spectral characteristic.

Note that the spectral characteristics of the ambient light source and the subject are estimated utilizing two images including an image obtained with the flash 31a on at a known intensity and an image obtained with the flash 31a off. For specific estimation methods for the spectral characteristics of the ambient light source and the subject, see, for example, “Practical Scene Illuminant Estimation via Flash/No-Flash Pairs, Cheng Lu and Mark S. Drew; School of Computing Science, Simon Fraser University, Vancouver, British Columbia, Canada V5A 1S6 {clu, mark}@cs.sfu.ca.”

The subject characteristic comparing section 35 reads, as a reference spectral characteristic, a spectral characteristic of a preferred state obtained when the skin is used as a subject and stored in the characteristic classification storage section 36, and compares the reference spectral characteristic with the subject spectral characteristic from the subject spectral characteristic estimating section 34 for evaluation. The subject characteristic comparing section 35 outputs an evaluation result to the recommended product selecting section 37. More specifically, the subject characteristic comparing section 35 determines a difference between the subject spectral characteristic and the reference spectral characteristic, compares a square root sum of the difference with a predetermined threshold, and outputs a comparison result to the recommended product selecting section 37 as an evaluation result.

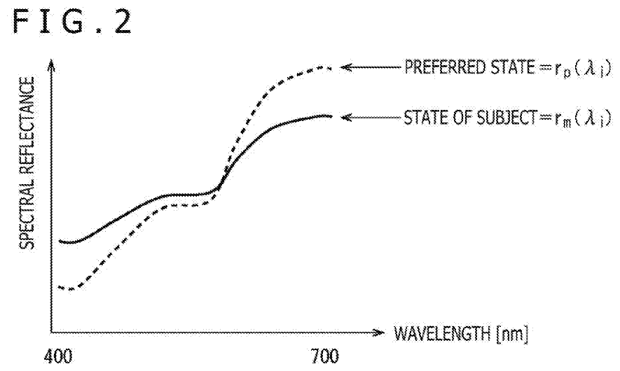

Specifically, as illustrated in FIG. 2, the subject characteristic comparing section 35 computes a difference dᵢ for each wavelength between the subject spectral characteristic (state of the subject=rₘ(λᵢ)) fed by the subject spectral characteristic estimating section 34 and indicated by a solid line and the reference spectral characteristic of the preferred state (preferred state=rₚ(λᵢ)) obtained when the skin is used as a subject and stored in the characteristic classification storage section 36 and indicated by a dotted line, as represented by Formula (1).

[Math. 1]

dᵢrₚ(λᵢ)−rₘ(λᵢ) (1)

As represented by Formula (2) below, the subject characteristic comparing section 35 compares a square root sum m of the difference dᵢ with a predetermined threshold th to evaluate the state of the skin of the user used as a subject, and outputs a comparison result as an evaluation result for the state of the skin of the user, while outputting both the subject spectral characteristic and the reference spectral characteristic of the preferred state to the recommended product selecting section 37.

[Math. 2]

Σ\sqrt{dᵢ²}>th (2)

Here, Σ√dᵢ² is the square root sum m of the difference dᵢ at a wavelength λᵢ.

Additionally, FIG. 2 illustrates the subject spectral characteristic (state of the subject=rₘ(λᵢ)) and the reference spectral characteristic (preferred state=rₚ(λᵢ)), and a horizontal axis indicates a wavelength, while a vertical axis indicates a spectral reflectance of the subject. As illustrated in FIG. 2, the subject spectral characteristic of the preferred state for the human skin varies depending on the site, and involves a darker red tint at a wavelength of approximately 700 nm and a paler blue tint at a wavelength of approximately 400 nm.

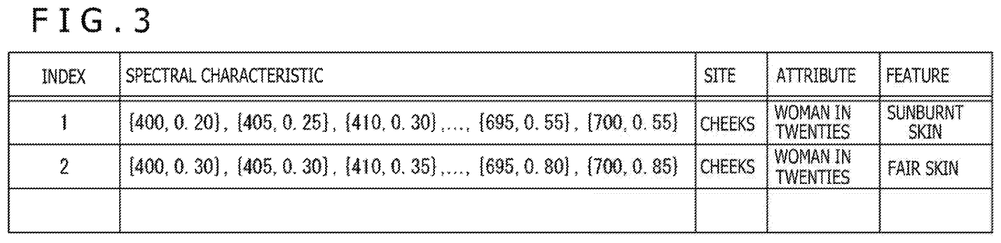

The characteristic classification storage section 36 stores the spectral characteristic of the preferred state as a reference spectral characteristic. Information related to the reference spectral characteristic of the preferred state is stored, for example, as a table as illustrated in FIG. 3. For the information related to the spectral characteristic, an index section, a spectral characteristic section, a site section, an attribute section, and a feature section are provided in this order from the left, and attributes including the site of the body, age, and sex and features such as a sunburnt skin and a fair skin are stored as illustrated in the table in FIG. 3. The preferred state may be any state recorded in the characteristic classification storage section 36 and can be determined by the user.

FIG. 3 illustrates examples with “cheeks” as a site and examples with “woman in twenties” as an attribute, and illustrates “sunburnt skin” and “fair skin” as features.

Additionally, in the spectral characteristic, for example, for an index=1, {400, 0.20}, {405, 0.25}, {410, 0.30}, . . . , {695, 0.55}, {700, 0.55} are indicated. In {A, B}, A represents the wavelength, and B represents the spectral reflectance. The subject characteristic comparing section 35 can restore a waveform as illustrated by a dotted line in FIG. 2 on the basis of information related to the spectral characteristic illustrated in FIG. 3.

Specifically, as illustrated in FIG. 3, for the reference spectral characteristic with the index=1, the spectral characteristic is “{400, 0.20}, {405, 0.25}, {410, 0.30}, . . . , {695, 0.55}, {700, 0.55},” the site is “cheeks,” the attribute is “woman in twenties,” and the feature is “sunburnt skin.”

Additionally, as illustrated in FIG. 3, for the reference spectral characteristic with the index=2, the spectral characteristic is “{400, 0.30}, {405, 0.30}, {410, 0.35}, . . . , {695, 0.80}, {700, 0.85},” the site is “cheeks,” the attribute is “woman in twenties,” and the feature is “fair skin.”

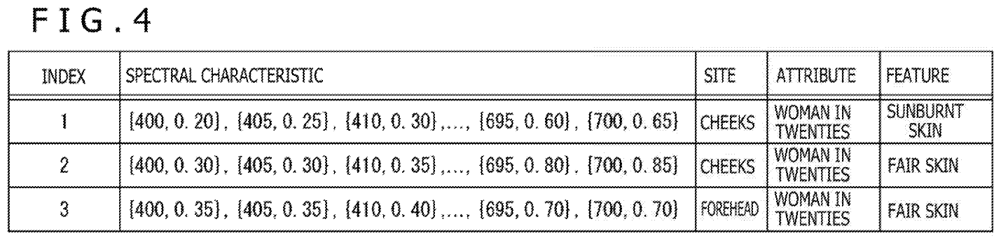

Furthermore, as illustrated in FIG. 4, the characteristic classification storage section 36 stores, as an average spectral characteristic, the average spectral characteristic of the subject corresponding to the average value for the subject spectral characteristics of N persons. FIG. 4 illustrates examples with “cheeks” and “forehead” as sites and examples with “woman in her twenties” as an attribute, and illustrates “sunburnt skin” and “fair skin” as features. Average subject spectral characteristics under respective conditions are stored.

Specifically, as illustrated in FIG. 4, for the average spectral characteristic with the index=1, the following are registered: the spectral characteristic is “{400, 0.20}, {405, 0.25}, {410, 0.30}, . . . , {695, 0.60}, {700, 0.65},” the site is “cheeks,” the attribute is “woman in twenties,” and the feature is “sunburnt skin.”

Specifically, for the average spectral characteristic with the index=2, the following are registered: the spectral characteristic is “{400, 0.30}, {405, 0.30}, {410, 0.35}, . . . , {695, 0.80}, {700, 0.85}”, the site is “cheeks,” the attribute is “woman in twenties,” and the feature is “fair skin.”

Furthermore, for the average spectral characteristic with the index=3, the following are registered: the spectral characteristic is “{400, 0.35}, {405, 0.35}, {410, 0.40}, . . . , {695, 0.70}, {700, 0.70},” the site is “forehead,” the attribute is “woman in twenties,” and the feature is “fair skin.”

The subject characteristic comparing section 35 may compute, as the threshold th, the difference square root sum m between the reference spectral characteristic of the preferred state and the average spectral characteristic and use the threshold th in evaluating the subject spectral characteristic. Additionally, the subject characteristic comparing section 35 may compute a variance σ from the reference spectral characteristic of the preferred state and the average spectral characteristic, determine the threshold th as m+ασ, and use the threshold in evaluating the subject spectral characteristic. Here, α is a parameter that can be adjusted by a designer.

In a comparison result of the difference square root sum m with the predetermined threshold th, which corresponds to the evaluation result for the state of the skin of the user, in a case where the difference square root sum m is larger than the predetermined threshold th, the recommended product selecting section 37 selects (identifies) a product (or an article) to be recommended from products (or articles) stored in the product storage section 38 on the basis of the subject spectral characteristic and the reference spectral characteristic of the preferred state, and outputs, to the output control section 39, product information related to the product corresponding to the selection result.

More specifically, the recommended product selecting section 37 computes a spectral characteristic rᵣ(λᵢ) of a suitable product state by multiplying the difference between the reference spectral characteristic rₚ(λᵢ) of the preferred state and the subject spectral characteristic rₘ(λᵢ) by a predetermined coefficient α, and adding a result of the multiplication to the reference spectral characteristic rₚ(λᵢ) of the preferred state on the basis of Formula (3).

[Math. 3]

rᵣ(λᵢ)=α(rₚ(λᵢ)−rₘ(λᵢ))+rₚ(λᵢ) (3)

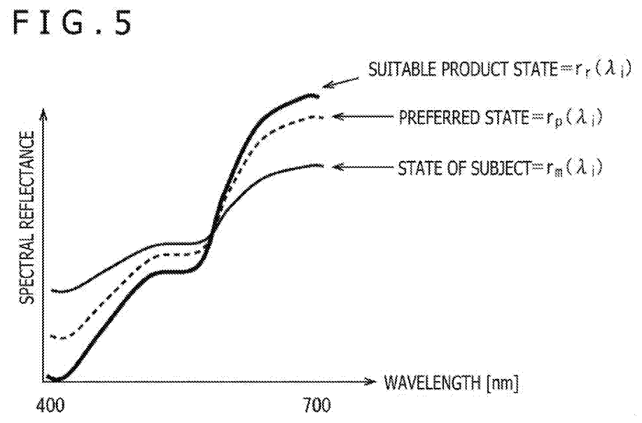

In other words, as illustrated in FIG. 5, the difference between the reference spectral characteristic rₚ(λᵢ) of the preferred state indicated by a dotted line and the subject spectral characteristic rₘ(λᵢ) indicated by a solid line is multiplied by the predetermined coefficient α, and the result of the multiplication is added to the reference spectral characteristic rₚ(λᵢ) of the preferred state, thus determining the spectral characteristic rᵣ(λᵢ) of the suitable product state indicated by a thick line. Here, the spectral characteristic of the suitable product state is, for example, the ideal spectral characteristic of the subject obtained when the product is applied to the skin, corresponding to the subject, in other words, the ideal spectral characteristic of the product applied to the subject. In contrast, the spectral characteristic of the preferred state (reference spectral characteristic) is, for example, the ideal subject spectral characteristic in a state before application of the product to the skin as a subject (state in which the product has not been applied). Accordingly, the spectral characteristic of the suitable product state can be said to be the spectral characteristic of the product expected to serve as the ideal spectral characteristic of the subject when applied to the skin, the spectral characteristic of the product being determined on the basis of the difference between the current estimated spectral characteristic of the subject and the (ideal) spectral characteristic (reference spectral characteristic) of the preferred state corresponding to a state before application of the product to the skin as a subject.

The recommended product selecting section 37 computes the sum Dⱼ of square of the difference between the spectral characteristic rᵣ(λᵢ) of the suitable product state and a spectral characteristic r_{dj}(λᵢ) in the product information registered in the product storage section 38 as represented by Formula (4) below.

[Math. 4]

Dⱼ=Σ(rᵣ(λᵢ)−r_{dj}(λᵢ))² (4)

Here, Dⱼ is the sum of square of the difference between the spectral characteristic rᵣ(λᵢ) of the suitable product state and the spectral characteristic r_{d} (λ₁) of a product registered in the product storage section 38.

Furthermore, the recommended product selecting section 37 searches the product storage section 38 for a product registered in the product storage section 38 and for which the sum Dⱼ of square of the difference between the spectral characteristic rᵣ(λᵢ) of the suitable product state and the spectral characteristic r_{dj}(λᵢ) of the product is minimized, selects the product as a product to be recommended, and outputs the product to the output control section 39, as represented by Formula (5) below.

$$\begin{matrix}[ Math . 5 ] & \\ Index of optimum product = arg ( {min}_{j} {D}_{j} ) & ( 5 )\end{matrix}$$

Here, arg(min Dⱼ) denotes a set of conditions under which the sum Dⱼ of square of the difference is minimized and indicates selection of index of the product registered in the product storage section 38 and for which the sum D of square of the difference between the spectral characteristic rᵣ(λᵢ) of the suitable product state and the spectral characteristic r_{dj}(λᵢ) of the product is minimized.

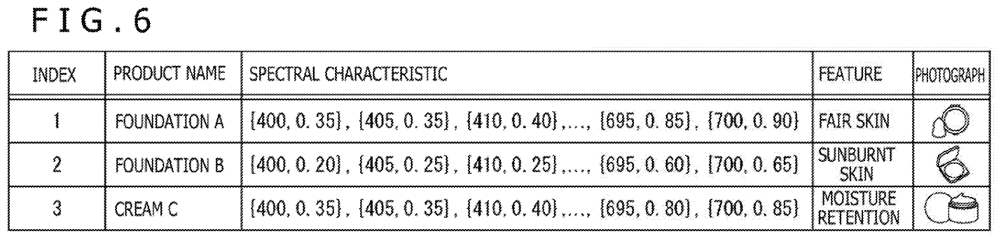

Here, the product storage section 38, for example, stores spectral characteristics for the indexes of the respective products as illustrated in FIG. 6.

In FIG. 6, an index section, a product name section, a spectral characteristic section, a feature section, and a product photograph section are provided in this order from the left, and product information corresponding to the respective sections is registered.

In FIG. 6, as product names, examples of “Foundation A,” “Foundation B,” and “Cream A” are illustrated in this order from the top, and as features, examples of “fair skin,” “sunburnt skin,” and “moisturization” are illustrated, with examples of product photographs displayed.

Specifically, in FIG. 6, the following are registered for product information with the index=1: the product name is “Foundation A,” the suitable spectral characteristic of the product is “{400, 0.35}, {405, 0.35}, {410, 0.40}, . . . , {695, 0.85}, {700, 0.90},” the feature is “fair skin,” with a corresponding product photograph registered.

Additionally, the following are registered for product information with the index=2: the product name is “Foundation B,” the suitable spectral characteristic of the product is “{400, 0.20}, {405, 0.25}, {410, 0.25}, . . . , {695, 0.60}, {700, 0.65},” the feature is “sunburnt skin,” with a corresponding product photograph registered.

Furthermore, the following are registered for product information with the index=3: the product name is “Cream C,” the suitable spectral characteristic of the product is “{400, 0.35}, {405, 0.35}, {410, 0.40}, . . . , {695, 0.80}, {700, 0.85},” the feature is “moisturization,” with a corresponding product photograph registered.

The inappropriate-light-source detecting section 41 compares the ambient-light-source spectral characteristic fed by the ambient-light-source estimating section 33 with the average spectral characteristic stored in the characteristic classification storage section 36 to determine whether the ambient light source is an inappropriate light source that is not suitable for estimating the subject spectral characteristic, and outputs a determination result to the output control section 39.

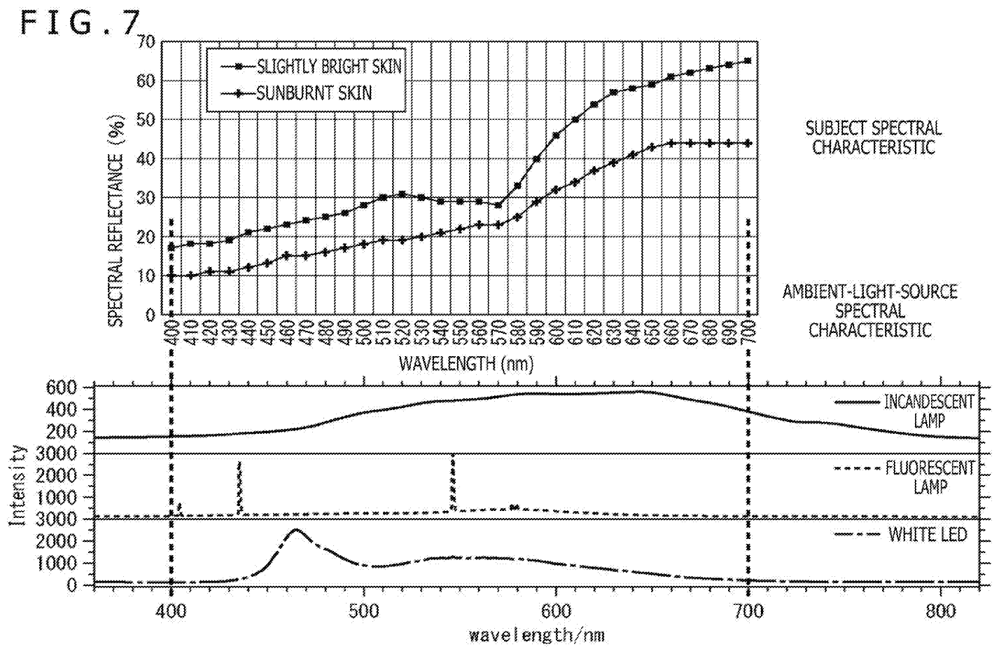

For example, as illustrated in an upper stage in FIG. 7, an intensity at which the ambient-light-source spectral characteristic is evenly distributed over the entire wavelength region is desirable for appropriately estimating the average spectral characteristic for a “slightly bright skin” and a “sunburnt skin” over the entire wavelength region.

Here, for appropriate estimation over the entire wavelength region, the ambient light source can be considered to be an appropriate ambient light source in a case where a certain intensity is distributed over the entire wavelength region as in the case of an incandescent lamp in the uppermost stage. However, for the ambient-light-source spectral characteristics of a fluorescent lamp and a white LED in the lower stage in FIG. 7, the intensity is obtained only at particular wavelength regions, and the ambient light sources are considered to be inappropriate ambient light sources.

Specifically, the fluorescent lamp has a pulsed characteristic and thus provides a sufficient intensity only within a pinpoint wavelength range near 435 nm and 545 nm. Additionally, the white LED provides a sufficient intensity only within a wavelength region from several tens of nm lower than 465 nm to several tens of nm higher than 465 nm.

More specifically, the inappropriate-light-source detecting section 41 executes a calculation in accordance with Formula (6) below to obtain the average intensity over the entire wavelength region to determine whether the ambient light source is an inappropriate light source or not depending on whether the average intensity is larger than a predetermined threshold or not.

$$\begin{matrix}[ Math . 6 ] & \\ \tilde{I} = \frac{1}{} \sum I ( λ ) & ( 6 )\end{matrix}$$

Here, Λ is the number of samples, and I(λ) is the intensity.

Additionally, the wavelength region may be divided into a plurality of regions, and whether the ambient light source is an inappropriate ambient light source or not may be determined depending on whether a sufficient intensity is obtained in each of the regions or not.

The entire wavelength region may be, for example, divided into four regions respectively ranging from 400 to 500 nm, from 500 to 570 nm, from 570 to 630 nm, and from 630 to 700 nm, and for example, computation may be executed in accordance with Formula (7) below to determine whether the ambient light source is an inappropriate ambient light source or not.

$$\begin{matrix}[ Math . 7 ] & \\ {\tilde{I}}_{i} = \frac{1}{{}_{i}} \sum I ( {λ}_{i} ) & ( 7 )\end{matrix}$$

Here, Λᵢ is the number of samples for each wavelength region, and I(λᵢ) is the intensity for each wavelength region.

Note that the upper stage in FIG. 7 illustrates the subject spectral characteristics for the “slightly bright skin” and the “sunburnt skin” and that the vertical axis indicates the spectral reflectance, while the horizontal axis indicates the wavelength. Additionally, note that the lower stage in FIG. 7 illustrates the ambient-light-source spectral characteristics for the incandescent lamp, fluorescent lamp, and the white LED from the top and that the vertical axis indicates the intensity, while the horizontal axis indicates the wavelength.

The output control section 39 acquires information related to a recommended product and fed by the recommended product selecting section 37 and a determination result from the inappropriate-light-source detecting section 41 indicating whether the ambient light source is an inappropriate light source or not. The output control section 39 controls the output section 40 to display product information related to the recommended product, and when the ambient light source is an inappropriate light source, presents warning information.

The output section 40 includes a display section 51, a vibrator 52, and a speaker 53, and is controlled to present various types of information.

The display section 51 is a display including an LCD (Liquid Crystal Display), an organic EL (Electronic Luminescent), or the like, and is controlled by the output control section 39 to display a predetermined image.

The vibrator 52 is controlled by the output control section 39, and vibrates the whole recommended product presenting apparatus 11, for example, in a case where a weight in an eccentric rotation shaft is rotated by a motor.

The speaker 53 is controlled by the output control section 39 to output a predetermined sound.

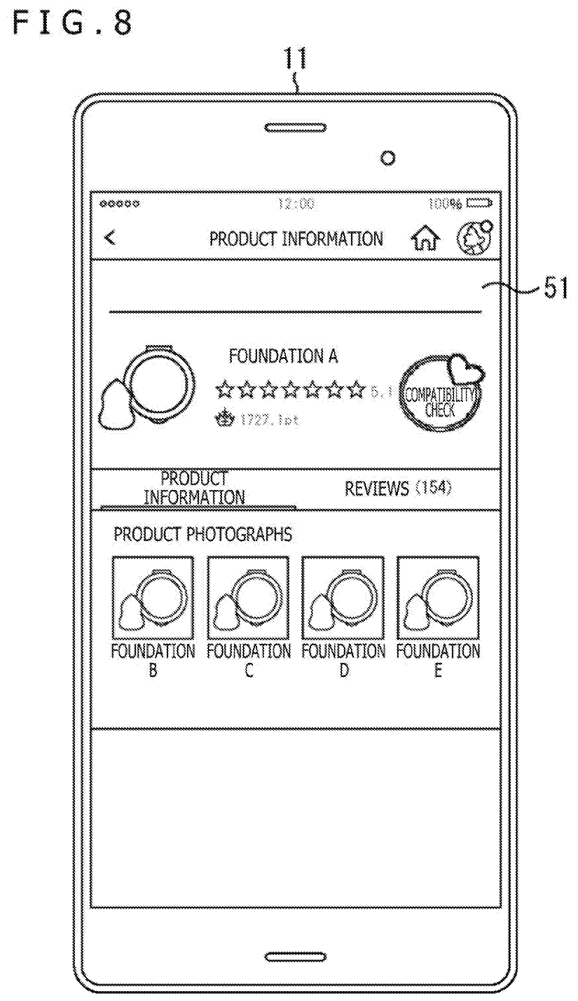

Specifically, the output control section 39 acquires information related to a recommended product and fed by the recommended product selecting section 37, and causes the display section 51 to display a product name and a product image as product information related to the product to be recommended, for example, as illustrated in FIG. 8.

FIG. 8 illustrates an example in which the display section 51 displays, as product information related to the recommended product, “Foundation A” in the upper stage as a product name and a product photograph on the right side of “Foundation A.” Additionally, in FIG. 8, below the product information related to “Foundation A,” “Foundation B” to “Foundation E” are displayed as related products, with respective product photographs displayed. Note that FIG. 8 illustrates an example with one recommended product but that a plurality of recommended products may be displayed.

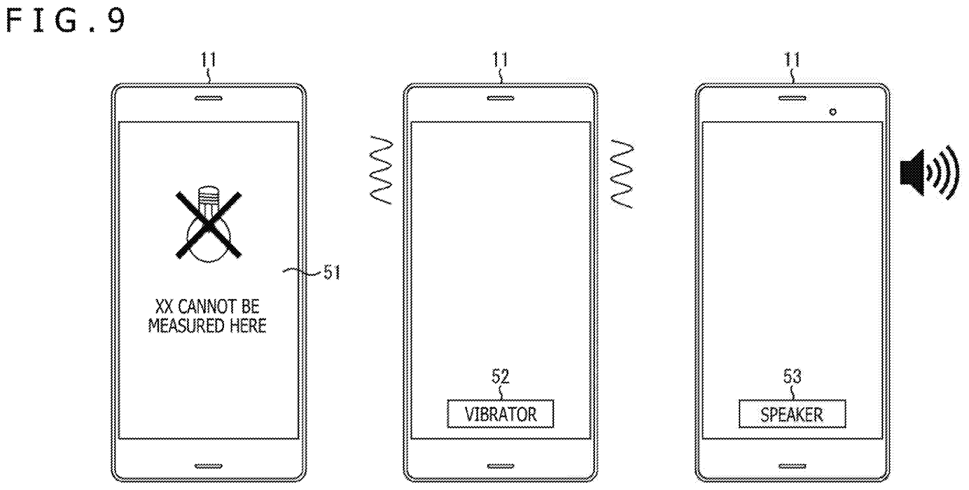

Additionally, in a case of being fed, from the inappropriate-light-source detecting section 41, with information indicating that the ambient-light-source spectral characteristic is inappropriate for acquiring the current subject spectral characteristic, the output control section 39, for example, as illustrated in a left portion of FIG. 9, generates a warning image with “XX CANNOT BE MEASURED HERE” to cause the display section 51 to display the warning image to warn a user that the ambient-light-source spectral characteristic is inappropriate. The warning is not limited to this, and for example, an image may be displayed that prompts another ambient light source to sufficiently capture an image in an environment. Additionally, “XX” in the left portion of FIG. 9 is, for example, a skin color or spots.

Furthermore, in a case of being fed, from the inappropriate-light-source detecting section 41, with information indicating that the ambient-light-source spectral characteristic is inappropriate for acquiring the current subject spectral characteristic, the output control section 39, for example, as illustrated in a central portion of FIG. 9, controls the vibrator 52 to vibrate the main body, warning the user that the ambient-light-source spectral characteristic is inappropriate.

Additionally, in a case of being fed, from the inappropriate-light-source detecting section 41, with information indicating that the ambient-light-source spectral characteristic is inappropriate, the output control section 39, for example, as illustrated in a right portion of FIG. 9, controls the speaker 53 to generate a warning sound to warn the user that the ambient-light-source spectral characteristic is inappropriate.

### <Product Recommendation Processing by Recommended Product Presenting Apparatus in FIG. 1>

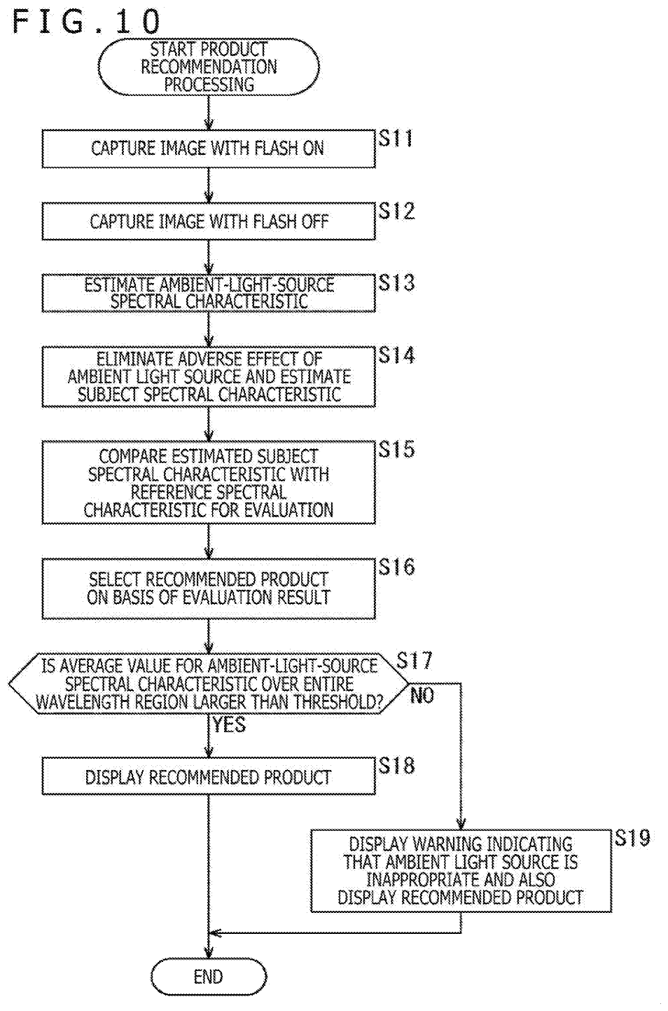

Now, with respect to a flowchart in FIG. 10, product recommendation processing by the recommended product presenting apparatus in FIG. 1 will be described.

In step S11, when the user operates the operation section 32 to instruct image capturing, the measurement section 31 controls the flash 31a, captures an image of a subject with the flash 31a on, and outputs the captured image to the ambient-light-source estimating section 33 and the subject spectral characteristic estimating section 34.

In step S12, the measurement section 31 captures an image of the subject with the flash 31a off, and outputs the captured image to the ambient-light-source estimating section 33 and the subject spectral characteristic estimating section 34.

In step S13, the ambient-light-source estimating section 33 uses the two images including the image with the flash 31a on and the image with the flash 31a off to estimate the spectral characteristic of the ambient light source, and outputs an estimation result to the subject spectral characteristic estimating section 34 and the inappropriate-light-source detecting section 41 as an ambient-light-source spectral characteristic.

In step S14, the subject spectral characteristic estimating section 34 uses the two images including the image with the flash 31a on and the image with the flash 31a off to eliminate the adverse effect of the ambient light source on the basis of the ambient-light-source spectral characteristic, then estimates the spectral characteristic of the subject in the image, and outputs an estimation result to the subject characteristic comparing section 35 as a subject spectral characteristic.

In step S15, the subject characteristic comparing section 35 compares the subject spectral characteristic, corresponding to the estimation result, with the reference spectral characteristic, corresponding to the spectral characteristic of the preferred state stored in the characteristic classification storage section 36, determines a comparison result as an evaluation result evaluating the state of the skin of the user, and outputs the subject spectral characteristic to the recommended product selecting section 37. At this time, the subject characteristic comparing section 35 outputs the subject spectral characteristic and the reference spectral characteristic of the preferred state to the recommended product selecting section 37 in conjunction with the evaluation result.

In step S16, on the basis of the evaluation result, the subject spectral characteristic, and the reference spectral characteristic of the preferred state, the recommended product selecting section 37 selects (identifies), as a recommended product, one of the products stored in the product storage section 38 that is to be recommended, and outputs the recommended product to the output control section 39.

Specifically, on the basis of the evaluation result, in a case where the comparison result of comparison between the difference square root sum m and the predetermined threshold th, corresponding to the evaluation result for the state of the skin of the user, indicates that the difference square root sum m is larger than the predetermined threshold th, the recommended product selecting section 37 executes calculation in accordance with Formula (3) using the subject spectral characteristic and the reference spectral characteristic of the preferred state to compute a suitable product state as described with reference to FIG. 5. Then, the recommended product selecting section 37 selects the index of a product with a spectral characteristic that minimizes the sum of square of the difference determined by Formula (4) and Formula (5), as indicated by the product information stored in the product storage section 38, that is, the product with the spectral characteristic most similar to the spectral characteristic of the suitable product state.

Note that the recommended product is selected in a case where the difference square root sum m between the subject spectral characteristic and the reference spectral characteristic is higher than the predetermined threshold th. Accordingly, the recommended product is not selected, for example, in a case where the difference square root sum m between the subject spectral characteristic and the reference spectral characteristic is smaller than the predetermined threshold th and where the comparison described with reference to FIG. 2 indicates that the state of the subject is close to or better than the preferred state. In this case, the recommended product selecting section 37 may output information indicating that the state of the skin of the user is of the preferred state, for example, “Your skin is in the preferred state.” Additionally, in a case where the state of the subject is close to or better than the preferred state, selection of the recommended product may be omitted as described above or a general product may be recommended.

In step S17, the inappropriate-light-source detecting section 41 determines whether the ambient light source is an inappropriate light source or not on the basis of whether or not the average intensity of the ambient-light-source spectral characteristic over the entire wavelength region is larger than a threshold corresponding to the average value of the subject spectral characteristic stored in the characteristic classification storage section 36 and the ambient light source is appropriate as an ambient light source with which the subject spectral characteristic is estimated, and outputs a determination result to the output control section 39.

In step S17, in a case where the average intensity of the ambient light source over the entire wavelength region is larger than the threshold corresponding to the average value of the subject spectral characteristic stored in the characteristic classification storage section 36 and where the ambient light source is determined to be appropriate as an ambient light source with which the subject spectral characteristic is estimated and not to be an inappropriate light source, the processing proceeds to step S18.

In step S18, the output control section 39 displays the product information related to the recommenced product on the display section 51 of the output section 40 to end the processing. Note that, for example, the recommended product is not selected in a case where the difference square root sum m between the subject spectral characteristic and the reference spectral characteristic is smaller than the predetermined threshold th and where the comparison described with reference to FIG. 2 indicates that the state of the subject is close to or better than the preferred state. In such a case, the recommended product selecting section 37 may output, to the output control section 39, information indicating that the state of the skin of the user is in the preferred state, for example, “Your skin is in the preferred state.” This allows the output control section 39 to display the information such as “Your skin is in the preferred state.” on the display section 51 of the output section 40 instead of the product information related to the recommended product.

On the other hand, in step S17, in a case where the average intensity of the ambient light source over the entire wavelength region is not larger than the threshold corresponding to the average value of the subject spectral characteristic stored in the characteristic classification storage section 36 and where the ambient light source is determined to be an inappropriate ambient light source with which the subject spectral characteristic is estimated and to be an inappropriate light source, the processing proceeds to step S19.

In step S19, the output control section 39 displays, on the display section 51 of the output section 40, information indicating that the ambient light source is an inappropriate light source, along with the information related to the recommended product. Additionally, the output control section 39 vibrates the vibrator 52 and causes the speaker 53 to output a predetermined sound, thus indicating information that the ambient light source is an inappropriate light source and ending the processing.

Note that it is sufficient that the warning that the ambient light source is inappropriate can be provided by at least one of display on the display section 51, vibration of the vibrator 52, or a sound from the speaker 53 and that not all of the configurations need to be used for warning. Additionally, as the warning method used in a case where the ambient light source is inappropriate, the user may be able to preset one of the display section 51, the vibrator 52, and the speaker 53 or a combination of the display section 51, the vibrator 52, and the speaker 53.

The above-described processing enables the skin of the user to be evaluated by capturing an image of the skin of the user, used as a subject, determining the subject spectral characteristic of the subject, corresponding to the skin of the user, with the adverse effect of the ambient light source taken into account, and comparing the subject spectral characteristic with the predetermined threshold. Furthermore, on the basis of evaluation corresponding to the comparison with the threshold, a product (or an article) bringing the state of the skin of the user into the suitable state can be recommended (identified) as a recommended product and presented.

Additionally, in the above-described example, according to the evaluation of the skin of the user, the product bringing the skin of the user into the suitable skin state is presented as a recommended product. However, the evaluation itself of the skin of the user may be presented. In other words, by capturing an image of the skin of the user, used as a subject and determining the subject spectral characteristic of the subject, corresponding to the skin of the user, with the adverse effect of the ambient light source taken into account, the following may be presented: the comparison result of comparison with the predetermined threshold set according to the average value of the subject spectral characteristic, the degree of a difference from the predetermined threshold, or the degree to which the state is good or bad.

Furthermore, in a case where the recommended product presenting apparatus 11 in FIG. 1 is considered to present information corresponding to the evaluation of the skin of the user, the presentation of the recommended product can be considered to be a piece of information included as the information presented as the evaluation of the skin of the user.

Accordingly, the information corresponding to the evaluation of the skin of the user may be information indicating the comparison result of comparison of the subject spectral characteristic of the subject corresponding to the skin of the user with the predetermined threshold set according to the average spectral characteristic indicated by the average value of the subject spectral characteristic, or information indicating the degree to which the state is good or bad according to the difference from the predetermined threshold. Additionally, the information corresponding to the evaluation of the skin of the user may be, for example, a comment such as “You are recommended to avoid further sunburn.” in a case where the sunburn has led to a generally low spectral reflectance. Thus, the information corresponding to the evaluation of the skin of the user may be presented along with or instead of presentation of the recommended product, or may be presented to encourage the user to take action in response to the evaluation.

Additionally, in the above-described example, the recommended product for achieving the suitable state depending on the state of the skin of the user, used as a subject, is presented. However, the subject is not limited to the color of the skin and any other subject may be evaluated on the basis of the color of the subject. For example, the evaluation may be made on the color of an image captured using, for example, any of hair, clothes, a food, and a paint as a subject, and on the basis of the evaluation, the following may be presented: a product providing hair with a suitable color, a clothing product with a suitable color, a recommended food with a suitable color, a recommended paint with a suitable color, and the like.

Additionally, depending on the evaluation result for the skin of the user, not only the recommended product such as a cosmetic product but also a service to be recommended may be presented. For example, esthetic therapy or healthcare action such as exercise may be recommended.

Furthermore, in the above-described example, the recommended product presenting apparatus 11 is a portable terminal such as a smartphone. However, the configuration except for the measurement section 31, the operation section 32, the output control section 39, and the output section 40 may be provided outside the main body, that is, the following may be configured outside the main body: the ambient-light-source estimating section 33, the subject spectral characteristic estimating section 34, the subject characteristic comparing section 35, the characteristic classification storage section 36, the recommended product selecting section 37, the product storage section 38, and the inappropriate-light-source detecting section 41. This may be implemented, for example, by a cloud server via a network.

This enables a processing load on the smartphone to be reduced. Additionally, in a case where a captured image of the skin of the user, used as a subject, is available, the skin of the user can be evaluated simply by transmitting the captured image to the recommended product presenting apparatus 11 implemented by a cloud server or the like.

Additionally, the above-described example uses the difference dᵢ between the subject spectral characteristic (state of the subject=rₘ(λᵢ)) and the reference spectral characteristic (preferred state=rₚ(λᵢ)) or the sum D of the spectral characteristic rᵣ(λᵢ) of the suitable product state and the spectral characteristic r_{dj}(λᵢ) of the product registered in the product storage section 38. However, these may be represented not only by the difference but also, for example, by a ratio.

### 2. Second Embodiment

In the example in the above description, the evaluation of the skin of the user is determined as the comparison result of comparison of the difference square root sum between the subject spectral characteristic based on the image of the skin of the user and the subject spectral characteristic of the preferred state, with the threshold based on the average spectral characteristic and the reference spectral characteristic of the preferred state, and a recommended product (or article) is selected (identified) and presented on the basis of the evaluation of the skin of the user.

However, the evaluation of the skin of the user is not limited to this, and for example, the wavelength region of the subject spectral characteristic may be divided into a plurality of regions, the subject spectral characteristic may be classified according to a combination of comparison results of comparison between the average value of the spectral reflectance and a threshold for each of the division wavelength regions, and a classification result may be used to evaluate the state of the skin of the user.

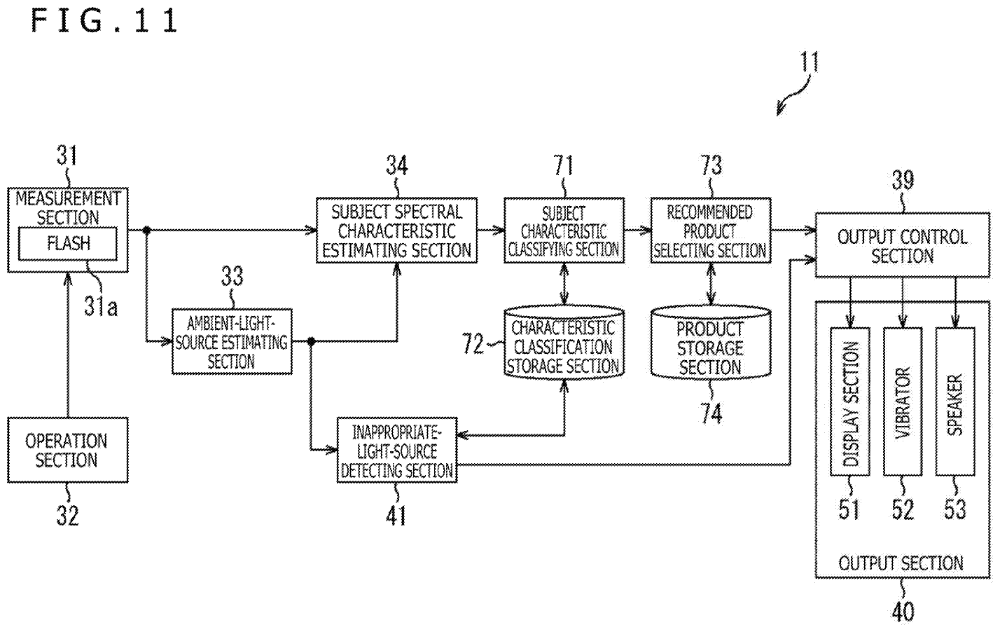

FIG. 11 illustrates a configuration example of the recommended product presenting apparatus 11 classifying the subject spectral characteristic according to a combination of comparison results of comparison between the average value of the spectral reflectance and the threshold for each wavelength region, and evaluating the state of the skin of the user on the basis of the classification result.

Those of the components of the recommended product presenting apparatus 11 in FIG. 11 which include the same functions as those of the corresponding components of the recommended product presenting apparatus 11 in FIG. 1 are denoted by the same reference numerals, and description of these components is appropriately omitted.

In other words, the recommended product presenting apparatus 11 in FIG. 11 differs from the recommended product presenting apparatus 11 in FIG. 1 in that a subject characteristic classifying section 71, a characteristic classification storage section 72, a recommended product selecting section 73, and a product storage section 74 instead of the subject characteristic comparing section 35, the characteristic classification storage section 36, the recommended product selecting section 37, and the product storage section 38.

The subject characteristic classifying section 71 outputs a classification result for the subject spectral characteristic corresponding to a combination of the comparison results of comparison between the average value of the spectral reflectance and the threshold for each wavelength region, to the recommended product selecting section 73 as evaluation of the state of the skin of the user.

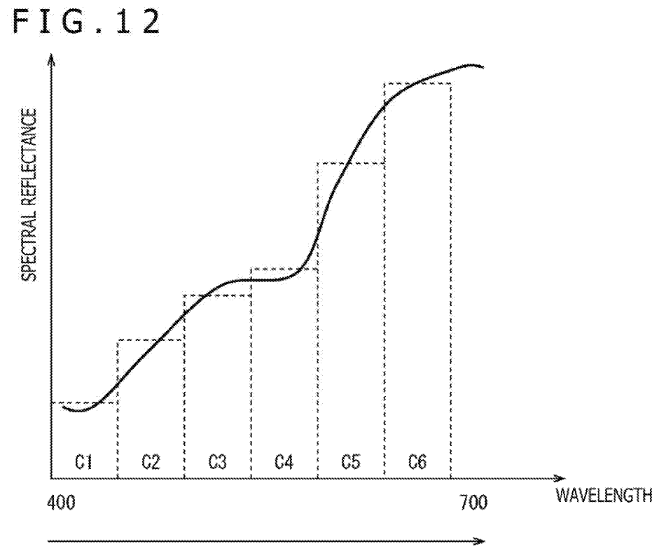

For example, in a case where the wavelength region of the subject spectral characteristic is divided into, for example, six wavelength regions, wavelength regions C1 to C6 as illustrated in FIG. 12, the subject characteristic classifying section 71 determines the average value of the subject spectral characteristic for each wavelength region as Formula (8) below.

$$\begin{matrix}[ Math . 8 ] & \\ \overline{{r}_{m}} = \frac{1}{c} \sum {r}_{m} ( {λ}_{i} ) & ( 8 )\end{matrix}$$

Here, c is the number of samples for each wavelength region, and rₘ(λᵢ) is the spectral reflectance at each wavelength λᵢ in each wavelength region Cm (m=1, 2, . . . 6).

Furthermore, on the basis of the comparison of the average value of the subject spectral characteristic with the threshold for each wavelength region, the subject characteristic classifying section 71 sets one of three values including a value smaller than the threshold, a value equivalent to the threshold, and a value larger than the threshold, and classifies the subject spectral characteristic according to a combination of three values for each wavelength region. That is, in this case, the subject spectral characteristic is classified into 3⁶=729 types. Then, the subject characteristic classifying section 71 reads, on the basis of the classification result for the subject spectral characteristic, an evaluation index stored in the characteristic classification storage section 72 in association with the classification result for the subject spectral characteristic, and outputs the evaluation index to the recommended product selecting section 73 as evaluation of the subject spectral characteristic.

The recommended product selecting section 73 reads, on the basis of the evaluation index, product information stored in the product storage section 74, and outputs the product information to the output control section 39. In other words, the product storage section 74 stores evaluation indexes in association with indexes of product information.

Note that the recommended product selecting section 37 in FIG. 1 selects product information indicating the minimum sum of square of the difference between the spectral characteristic of the product and the spectral characteristic of the suitable product state determined from the subject spectral characteristic and the reference spectral characteristic, and outputs the product information as a recommended product. In contrast, the recommended product selecting section 73 selects (identifies) the product information (or article information) with the index stored in the product storage section 74 in association with the evaluation index corresponding to the classification result for the subject spectral characteristic, and outputs the product information as a recommended product (or recommended article).

In other words, the recommended product selecting section 37 in FIG. 1 can be said to select, as a recommended product, product information including a spectral characteristic most similar to the spectral characteristic of the suitable product state substantially determined on the basis of the subject spectral characteristic and the reference spectral characteristic.

Here, the spectral characteristic of the suitable product state is determined from the subject spectral characteristic, and the subject spectral characteristic is also determined from the spectral characteristic of the suitable product state. Similarly, the evaluation index corresponding to the classification result for the subject spectral characteristic is determined from the spectral characteristic of the suitable product state for the corresponding subject spectral characteristic, and the spectral characteristic of the suitable product state is determined from the evaluation index corresponding to the classification result for the subject spectral characteristic.

Accordingly, in the product storage section 74, the index of product information for which the spectral characteristic of the suitable product state is most similar to the spectral characteristic in the product information is registered in association with the evaluation index.

The recommended product selecting section 37 compares the subject spectral characteristic with the predetermined threshold th to evaluate the state of the skin of the user, and on the basis of the evaluation result, selects, as a recommended product, a product with a spectral characteristic most similar to the spectral characteristic of the suitable product state.

In contrast, the recommended product selecting section 73 classifies the subject spectral characteristic into the evaluation index to evaluate the state of the skin of the user, and selects, as a recommended product, a product with the index of product information including the spectral characteristic of the suitable product state identified by the evaluation index corresponding to an evaluation result.

In other words, the recommended product selecting sections 37 and 73 differ from each other in that the acquired evaluation results for the recommended product selecting sections 37 and 73 are the subject spectral characteristic and the evaluation index, respectively, but can be said to execute substantially the same processing in that both recommended product selecting sections select product information including a spectral characteristic most similar to the spectral characteristic of the suitable product state obtained from the subject spectral characteristic and the evaluation index.

### <Product Recommendation Processing by Recommended Product Presenting Apparatus in FIG. 11>

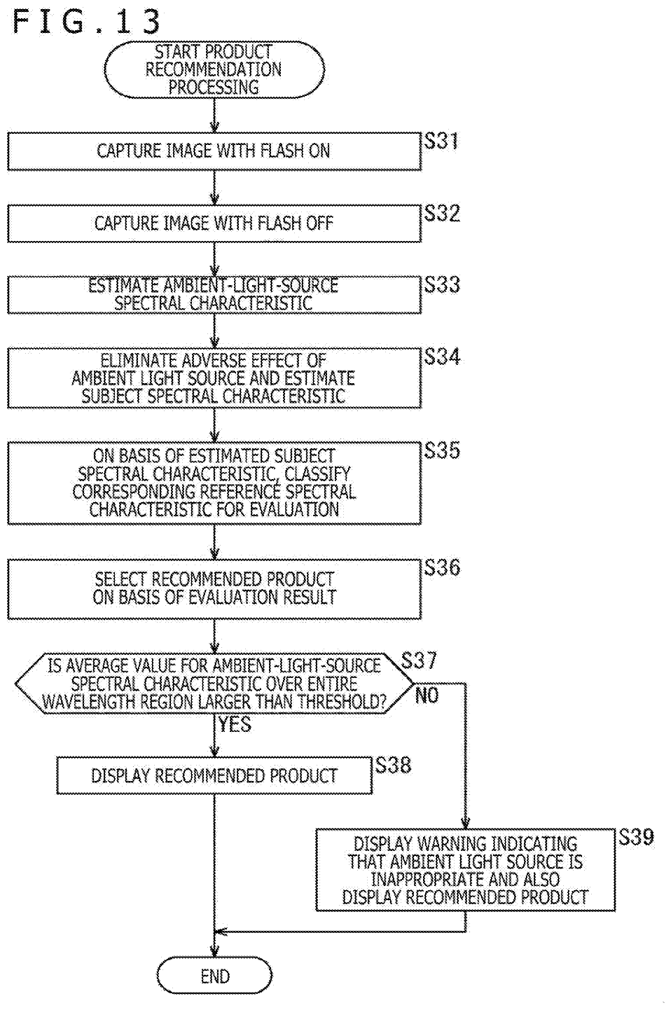

Now, with reference to a flowchart in FIG. 13, product recommendation processing by the recommended product presenting apparatus in FIG. 11 will be described. Note that processing in steps S31 to S34 and S37 to S39 in the flowchart in FIG. 13 is similar to the processing in steps S11 to S14 and S17 to S19 in FIG. 10 and thus that description of the processing is omitted.

Specifically, in step S35, on the basis of a classification result for a subject spectral characteristic corresponding to a combination of comparison results of comparison between the average value of the spectral reflectance and a threshold for each wavelength region, the subject characteristic classifying section 71 reads, from the characteristic classification storage section 72, information related to the corresponding evaluation index, and outputs the information to the recommended product selecting section 73 as an evaluation result of the skin of the user.

In step S36, on the basis of information with the evaluation index corresponding to the classification result, the recommended product selecting section 73 reads, from the product storage section 74, product information with the index registered in association with the evaluation index, selects (identifies) the product information as the recommended product (or recommended article) to be recommended, and outputs the recommended product to the output control section 39.

The above-described processing enables the skin of the user to be evaluated by capturing an image of the skin of the user, used as a subject, determining the subject spectral characteristic of the subject, corresponding to the skin of the user, with the adverse effect of the ambient light source taken into account, classifying the subject spectral characteristic, and outputting an evaluation index corresponding to a classification result. Furthermore, on the basis of the evaluation index set according to the classification result, a product (or article) with an index registered in association with the evaluation index as product information bringing the state of the skin of the user into the suitable product state can be caused to be selected (identified) and presented.

### 3. Third Embodiment

In the example described above, on the basis of an image captured by the measurement section 31, the skin of the user is evaluated utilizing the ambient-light-source spectral characteristic and the subject spectral characteristic. On the basis of the evaluation, a product achieving the suitable product state is presented as a recommended product, and warning information is presented when the ambient light source is inappropriate.

However, an inappropriate ambient light source may preclude the skin of the user from being appropriately evaluated, and the use of a recommended product presented on the basis of an inappropriate evaluation may prevent appropriate effects from being exerted. Thus, on the basis of an estimated ambient-light-source spectral characteristic, items enabling the skin of the user to be appropriately evaluated may be presented such that the user can select from the items, allowing presentation of a recommended product based on appropriate evaluation for the selected item.

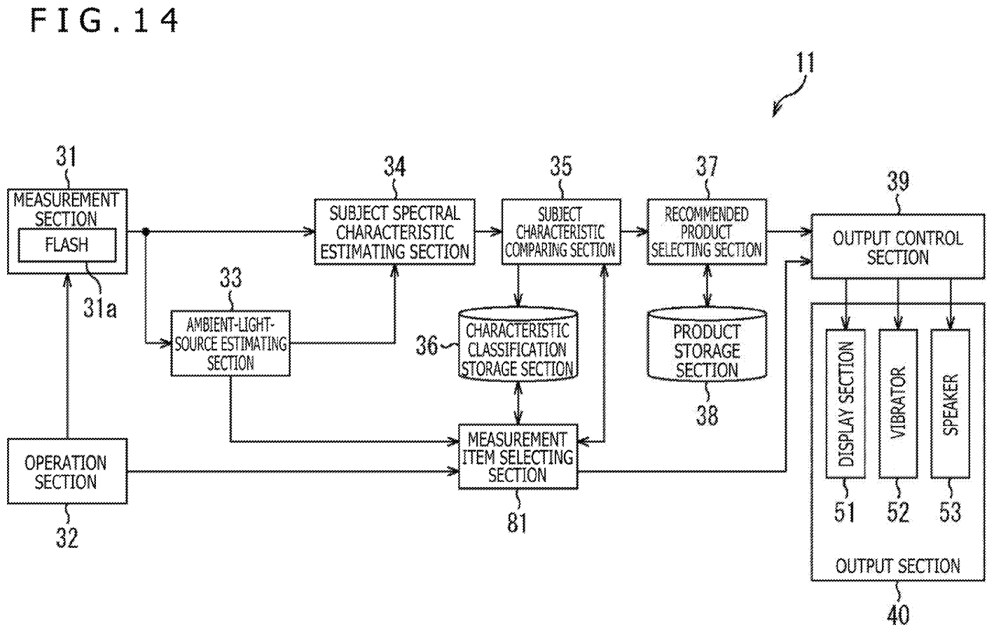

FIG. 14 illustrates a configuration example of the recommended product presenting apparatus 11 that presents, on the basis of the estimated ambient-light-source spectral characteristic, the items enabling the skin of the user to be appropriately evaluated such that the user can select from the items, allowing presentation of the recommended product based on appropriate evaluation for the selected item.

Those of the components of the recommended product presenting apparatus 11 in FIG. 14 which include the same functions as those of the corresponding components of the recommended product presenting apparatus 11 in FIG. 1 are denoted by the same reference numerals, and description of these components is appropriately omitted.

In other words, the recommended product presenting apparatus 11 in FIG. 14 differs from the recommended product presenting apparatus 11 in FIG. 1 in that a measurement item selecting section 81 is provided instead of the inappropriate-light-source detecting section 41.

The measurement item selecting section 81 compares the ambient-light-source spectral characteristic fed by the ambient-light-source estimating section 33 with the subject spectral characteristic stored in the characteristic classification storage section 36 to extract measurement items suitable for measurement, outputs the measurement items to the output control section 39 to cause the output control section 39 to display a selection screen for a measurement item suitable for measurement. Then, in a case where the user operates the operation section 32 to select a measurement item on the basis of the selection screen, the measurement item selecting section 81 outputs the selected selection item to the subject characteristic comparing section 35.

The subject characteristic comparing section 35 outputs, as an evaluation result for the state of the skin of the user, a comparison result of comparison of the difference square root sum between the reference spectral characteristic and the subject spectral characteristic for the user in the wavelength region corresponding to the selection item with a threshold utilizing the difference square root sum between the average spectral characteristic and the reference spectral characteristic.

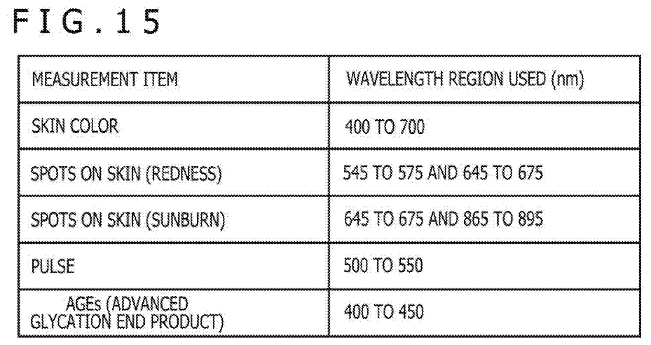

More specifically, for example, as illustrated in FIG. 15, in a case where the measurement item is a skin color, the wavelength region used ranges from 400 to 700 nm. In a case where the measurement item is spots (redness) on the skin, the wavelength region used ranges from 545 to 575 nm and from 645 to 675 nm. In a case where the measurement item is spots (sunburn) on the skin, the wavelength region used ranges from 645 to 675 nm and from 845 to 875 nm. In a case where the measurement item is a pulse, the wavelength region used ranges from 500 to 550 nm. In a case where the measurement item is AGEs (Advanced Glycation End Products), the wavelength region used ranges from 400 to 450 nm.

The subject characteristic comparing section 35 uses, according to the measurement item fed by the measurement item selecting section 81, the subject spectral characteristic of the corresponding wavelength region to compare the subject spectral characteristic with the reference spectral characteristic, and on the basis of the comparison result, evaluates the state of the skin of the user and outputs the state to the recommended product selecting section 37.

The recommended product selecting section 37 determines the suitable product state for the wavelength region corresponding to the measurement item on the basis of the subject spectral characteristic and the preferred state of the reference spectral characteristic, selects a product to be recommended from the products stored in the product storage section 38, and outputs, to the output control section 39, product information related to the product corresponding to the selection result.

### <Product Recommendation Processing by Recommended Product Presenting Apparatus in FIG. 14>

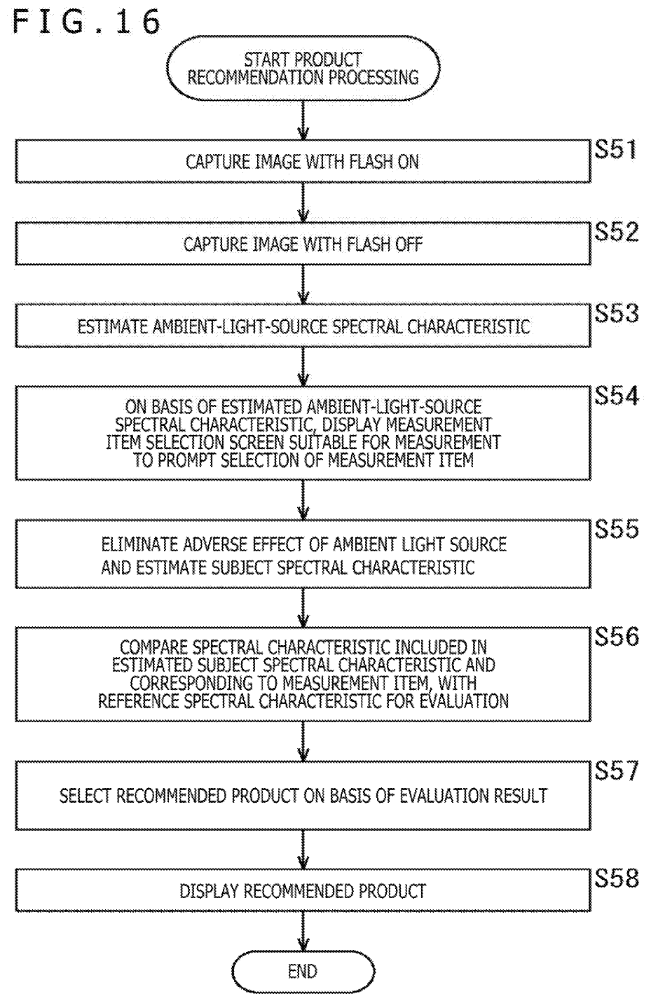

Now, with reference to a flowchart in FIG. 16, product recommendation processing by the recommended product presenting apparatus in FIG. 14 will be described.

The processing in steps S51 to S53 causes the measurement section 31 to capture an image of the skin of the user, used as a subject, and on the basis of the captured image, the ambient-light-source estimating section 33 estimates and outputs an ambient-light-source spectral characteristic to the subject spectral characteristic estimating section 34 and the measurement item selecting section 81.

In step S54, on the basis of the ambient-light-source spectral characteristic, the measurement item selecting section 81 selects and outputs a measurement item suitable for measurement to the output control section 39.

More specifically, in a case where, for example, in a portion of the ambient-light-source spectral characteristic from 400 to 700 nm, the difference square root sum m is higher than the threshold, the skin color is selected as a measurement item suitable for measurement as described with reference to FIG. 15. Additionally, similarly, in a case where, in portions of the ambient-light-source spectral characteristic from 545 to 575 nm and from 645 to 675 nm, the difference square root sum m is higher than the threshold, spots (redness) on the skin is selected as a measurement item suitable for measurement as described with reference to FIG. 15.

Furthermore, in a case where, in portions of the ambient-light-source spectral characteristic from 645 to 675 nm and from 845 to 875 nm, the difference square root sum m is higher than the threshold, spots (sunburn) on the skin are selected as a measurement item suitable for measurement. Additionally, in a case where, in a portion of the ambient-light-source spectral characteristic from 500 to 550 nm, the difference square root sum m is higher than the threshold, the pulse is selected as a measurement item suitable for measurement. Furthermore, in a case where, in a portion of the ambient-light-source spectral characteristic from 400 to 450 nm, the difference square root sum m is higher than the threshold, AGEs (Advanced Glycation End Products) are selected as a measurement item suitable for measurement.

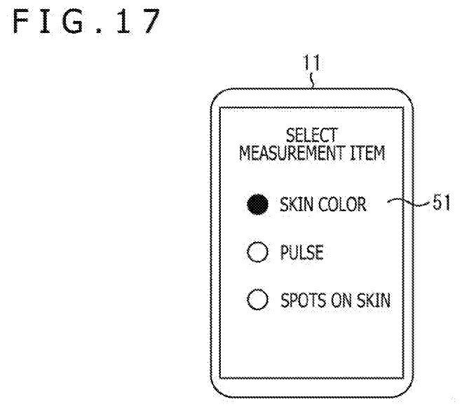

The output control section 39 generates, for example, a selection screen as illustrated in FIG. 17 on the basis of information related to measurement items suitable for measurement and causes the display section 51 to display the selection screen.

In FIG. 17, “SELECT MEASUREMENT ITEM” is indicated in the uppermost stage, with measurement items suitable for measurement indicated below “SELECT MEASUREMENT ITEM.” One of the measurement items suitable for measurement can be selected by operating, via the operation section 32, circle buttons on the left side. In the example illustrated in FIG. 17, measurement items are illustrated in the order of “SKIN COLOR,” “PULSE,” and “SPOTS ON SKIN” from the top of the figure, the button in the uppermost stage is colored, and “SKIN COLOR” has been selected as a measurement item.

When a measurement item selection screen is displayed as illustrated in FIG. 17 and the user operates the operation section 32 to select a measurement item, the measurement item selecting section 81 outputs, to the subject characteristic comparing section 35, the selected measurement item and the corresponding information related to the wavelength region.

In step S55, the subject spectral characteristic estimating section 34 estimates the subject spectral characteristic and outputs an evaluation result to the subject characteristic comparing section 35.

In step S56, the subject characteristic comparing section 35 determines the difference between the subject spectral characteristic and the reference spectral characteristic for the wavelength region required to measure the selected measurement item to determine the difference square root sum m, compares the difference square root sum m with the threshold th to evaluate the skin of the user, and outputs an evaluation result for the subject spectral characteristic and information related to the selected measurement item, to the recommended product selecting section 37 along with the comparison result of the comparison with the threshold th, corresponding to an evaluation result.

In step S57, on the basis of the evaluation result for the subject spectral characteristic and the information related to selected measurement item, along with the comparison result of comparison of the difference square root sum m with the threshold th for the wavelength region required to measure the selected measurement item, the recommended product selecting section 37 selects, as a recommended product, a product to be recommended from the product information stored in the product storage section 38 and outputs, to the output control section 39, information related to the selected recommended product. Note that details of the method for selecting a recommended product are similar to the details of the method for selecting a recommended product executed by the recommended product presenting apparatus 11 in FIG. 1 except for the use of only the information related to the wavelength region required to measure the selected measurement item and that the description of the details is omitted.

In step S58, the output control section 39 causes the display section 51 to display the information related to the recommended product.

The above-described processing enables the appropriate evaluation of the skin of the user based on the ambient light source to be achieved by capturing an image of the skin of the user, used as a subject, displaying the measurement item selection screen including, as choices, only measurement items suitable for measurement with the ambient light source to allow the user to select one of the choices, using the subject spectral characteristic required for the selected measurement item to compare the subject spectral characteristic with the predetermined threshold. Additionally, on the basis of the appropriate evaluation result, a product bringing the state of the skin of the user into the suitable state can be appropriately selected (identified) and presented as a recommended product (or recommended article). Note that, for the measurement item suitable for measurement with the ambient light source, the skin of the user may be appropriately evaluated on the basis of the ambient light source by displaying the measurement item and automatically using the subject spectral characteristic of the measurement item suitable for measurement to compare the subject spectral characteristic with the predetermined threshold. This enables the evaluation of the skin to be quickly presented without a need to wait for display of the measurement item selection screen and selection by the user.

### 4. Fourth Embodiment

In the above-described example, the ambient-light-source spectral characteristic is estimated each time, and the product recommendation processing based on the subject spectral characteristic is implemented. However, in a case where the user limits the use to a somewhat limited environment, the ambient-light-source spectral characteristic measured once may be stored for reuse.

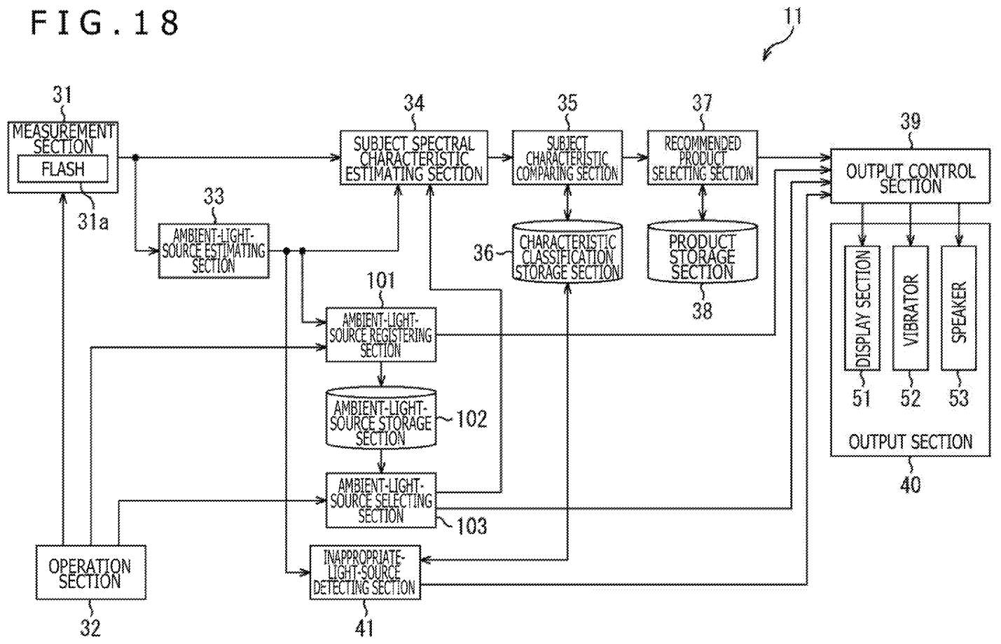

FIG. 18 is a configuration example of the recommended product presenting apparatus 11 that stores the ambient-light-source spectral characteristic for reuse. Note that those of the components of the recommended product presenting apparatus 11 in FIG. 18 which include the same functions as those of the corresponding components of the recommended product presenting apparatus 11 in FIG. 1 are denoted by the same reference numerals, and description of these components is appropriately omitted.

Specifically, the recommended product presenting apparatus 11 in FIG. 18 differs from the recommended product presenting apparatus 11 in FIG. 1 in that an ambient-light-source registering section 101, an ambient-light-source storage section 102, and an ambient-light-source selecting section 103.

When fed with a new ambient-light-source spectral characteristic from the ambient-light-source estimating section 33, the ambient-light-source registering section 101 causes, via the output control section 39, the display section 51 to display an image that inquires of the user as to whether to register the ambient-light-source spectral characteristic or not. Then, when the user operates, in response to the display, the operation section 32 to instruct registration, the ambient-light-source registering section 101 registers the ambient-light-source spectral characteristic in the ambient-light-source storage section 102 in association with the current position information.

Note that, for the current position information, the user may operate the operation section 32 to input information such as “user's room,” “washroom,” or “lavatory” as information identifying the location. Additionally, the current position information may be registered in association with position information including the latitude and longitude on the earth detected utilizing a GPS (Global Positioning System) not illustrated or the like.

When the measurement section 31 captures an image, the ambient-light-source selecting section 103 causes, via the output control section 39, the display section 51 to display an image that inquires which of the ambient-light-source spectral characteristics registered in the ambient-light-source storage section 102 is to be utilized. When one of the ambient-light-source spectral characteristics is selected, information related to the selected ambient-light-source spectral characteristic is read and output to the subject spectral characteristic estimating section 34.

### <Product Recommendation Processing by Recommendation Processing Apparatus in FIG. 18>

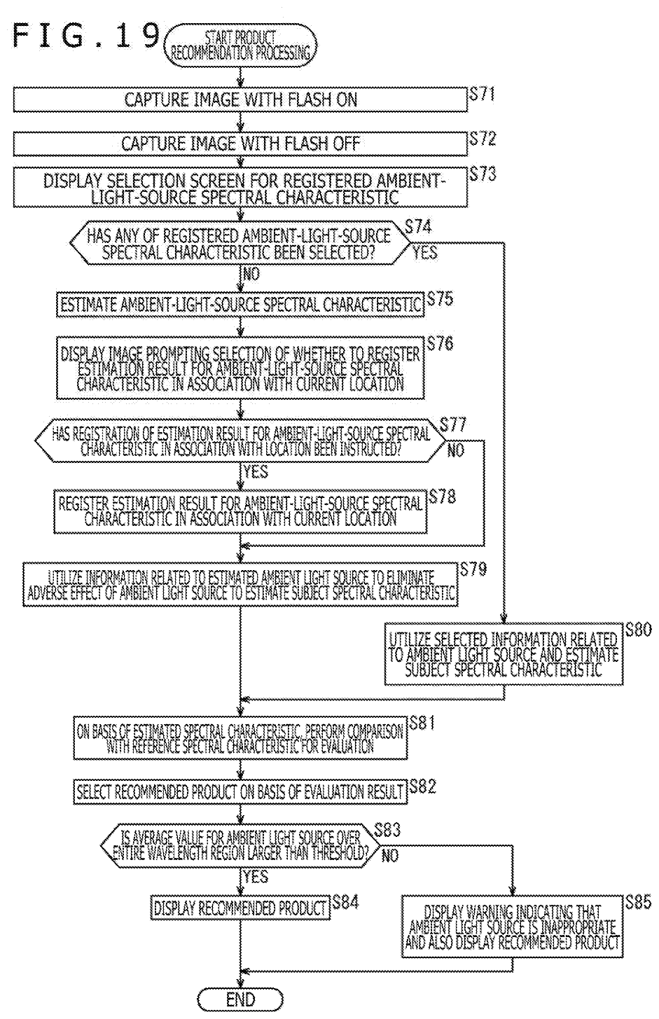

Now, with a flowchart in FIG. 19, product recommendation processing by the recommended product presenting apparatus 11 in FIG. 18 will be described.

In steps S71 and S72, the measurement section 31 captures an image with the flash 31a on and an image with the flash 31a off, and outputs the images to the ambient-light-source estimating section 33 and the subject spectral characteristic estimating section 34.

In step S73, the ambient-light-source selecting section 103 causes, via the output control section 39, the display section 51 to display a selection image for selection from pieces of information related to the ambient-light-source spectral characteristic stored in the ambient-light-source storage section 102 or for selection of non-use of the registered ambient-light-source spectral characteristics.

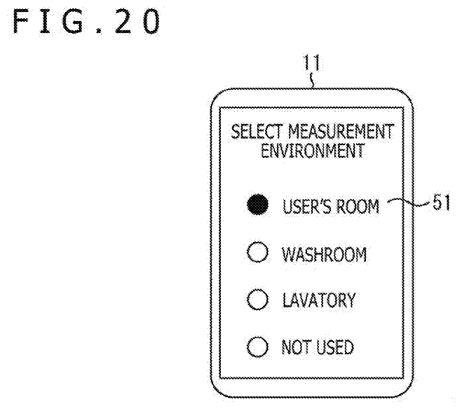

FIG. 20 illustrates an example of display of a selection image on the display section 51. In the uppermost stage, “select measurement environment” is indicated, and below the indication, “user's room,” “washroom,” “lavatory,” “not used” are displayed in this order from the top of the figure as choices for locations registered in association with the ambient-light-source spectral characteristics registered in the ambient-light-source storage section 102, with a circle selection button displayed on the left side of each of the choices.

One of selection buttons is operated by the operation section 32 to select the corresponding choice.

In step S74, the ambient-light-source selecting section 103 determines whether any of the registered ambient-light-source spectral characteristics has been selected by operating the operation section 32.

In step S74, in a case where, for example, “not used” is selected with none of the registered ambient-light-source spectral characteristics selected, the processing proceeds to step S75.

In step S75, the ambient-light-source estimating section 33 estimates the ambient-light-source spectral characteristic from two images including an image with the flash 31a on and an image with the flash 31a off, and outputs an evaluation result to the subject spectral characteristic estimating section 34, the inappropriate-light-source detecting section 41, and the ambient-light-source registering section 101.

In step S76, the ambient-light-source registering section 101 causes, via the output control section 39, the display section 51 to display an image inquiring whether to register the ambient-light-source spectral characteristic estimated in the corresponding place in association with the current location of the display section 51.

In step S77, the ambient-light-source registering section 101 determines whether registration of the ambient-light-source spectral characteristic estimated in the corresponding place in the ambient-light-source storage section 102 in association with the location has been instructed by operating the operation section 32.

In step S77, in a case where registration of the ambient-light-source spectral characteristic estimated in the corresponding place in the ambient-light-source storage section 102 in association with the location has been instructed, the processing proceeds to step S78.

In step S78, the ambient-light-source registering section 101 registers the ambient-light-source spectral characteristic estimated in the corresponding place, in the ambient-light-source storage section 102 in association with information related to the current location.

In step S77, in a case where no instruction to register the ambient-light-source spectral characteristic has been provided, the processing in step S78 is skipped.

In step S79, the subject spectral characteristic estimating section 34 uses the ambient-light-source spectral characteristic estimated in the corresponding place to eliminate the adverse effect of the ambient light source, estimates the spectral characteristic of the subject in the image using the two images including the image with the flash 31a on and the image with the flash 31a off, and outputs an estimation result to the subject characteristic comparing section 35 as a subject spectral characteristic.

Note that, in a case where any of the ambient-light-source spectral characteristics registered in the ambient-light-source storage section 102 is selected, the processing proceeds to step S80.

In step S80, the subject spectral characteristic estimating section 34 uses the selected ambient-light-source spectral characteristic to eliminate the adverse effect of the ambient light source, estimates the spectral characteristic of the subject in the image using the two images including the image with the flash 31a on and the image with the flash 31a off, and outputs an estimation result to the subject characteristic comparing section 35 as a subject spectral characteristic.

Note that the processing in steps S81 to S85 is similar to the processing in steps S15 to S19 in the flowchart in FIG. 10 and that description of the processing is omitted.

Specifically, in a case where any of the registered ambient-light-source spectral characteristics is selected, the subject spectral characteristic is estimated with the adverse effect of the ambient light source eliminated by utilizing the selected ambient-light-source spectral characteristic. In a case where none of the registered ambient-light-source spectral characteristics is selected, the subject spectral characteristic is estimated with the adverse effect of the ambient light source eliminated by utilizing the ambient-light-source spectral characteristic estimated in the corresponding place.

As a result, the ambient-light-source spectral characteristic of the location registered once need not be estimated and can be reused simply by being read. This allows for a reduction in load related to processing for estimating the ambient-light-source spectral characteristic, enabling an increase in processing speed.

### 5. Fifth Embodiment

In the above-described example, the images captured by the measurement section 31 capturing images of the subject are used to estimate both the subject spectral characteristic and the ambient-light-source spectral characteristic. However, different measurement sections may be provided with the subject spectral characteristic and the ambient-light-source spectral characteristic to allow the ambient-light-source spectral characteristic to be more accurately estimated.

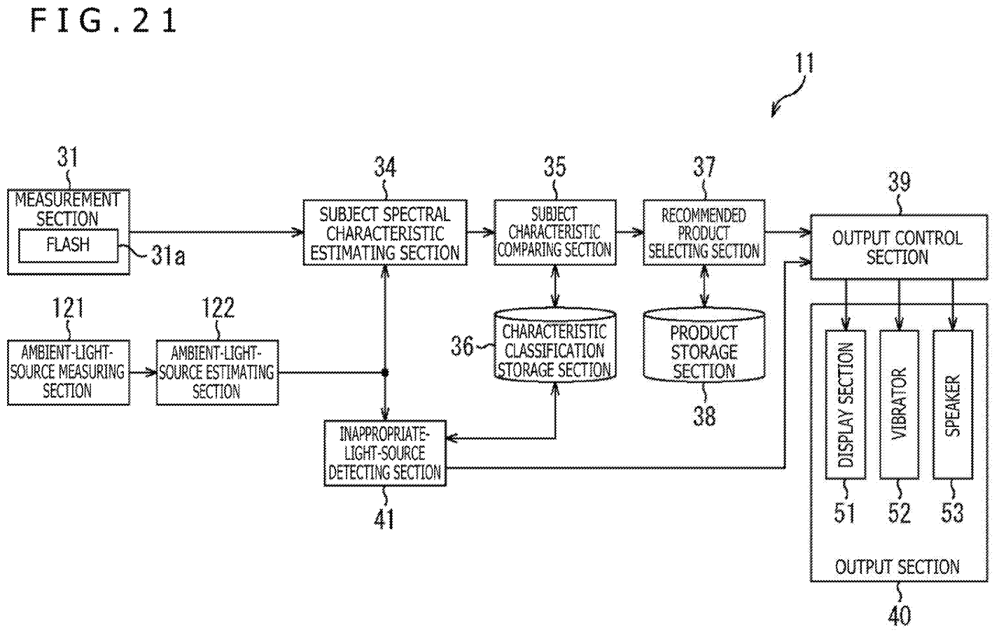

FIG. 21 illustrates a configuration example of the recommended product presenting apparatus 11 provided with an ambient-light-source measuring section for capturing an image for estimation of the ambient-light-source spectral characteristic in addition to the measurement section 31 capturing an image for estimation of the subject spectral characteristic.

Note that those of the components of the recommended product presenting apparatus 11 in FIG. 21 which include the same functions as those of the corresponding components of the recommended product presenting apparatus 11 in FIG. 1 are denoted by the same reference numerals, and description of these components is appropriately omitted.

Specifically, the recommended product presenting apparatus 11 in FIG. 21 differs from the recommended product presenting apparatus 11 in FIG. 1 in that the recommended product presenting apparatus 11 in FIG. 21 is newly provided with an ambient-light-source measuring section 121 that captures an image for estimation of the ambient-light-source spectral characteristic and an ambient-light-source estimating section 122.

The ambient-light-source measuring section 121 basically has the same configuration as that of the measurement section 31, but captures an image of a direction in which an image suitable for measurement of an ambient light source that may be used as an ambient light source can be captured and outputs the captured image to the ambient-light-source estimating section 122. Desirably, the ambient-light-source measuring section 121 is provided in a wearable terminal and configured to appropriately capture an image of a light source that may be used as an ambient light source.

The ambient-light-source estimating section 122 estimates, on the basis of an image captured by the ambient-light-source measuring section 121, the type and the spectral characteristic of a light source as described in, for example, Masaharu Tominaga “Computer Vision and Spectral Reflectance Estimation (Japanese Journal of Applied Physics), 1997, Vol. 26, No. 12,” and outputs an estimation result to the subject spectral characteristic estimating section 34 and the inappropriate-light-source detecting section 41 as an ambient-light-source spectral characteristic.

### <Product Recommendation Processing by Recommended Product Presenting Apparatus in FIG. 21>

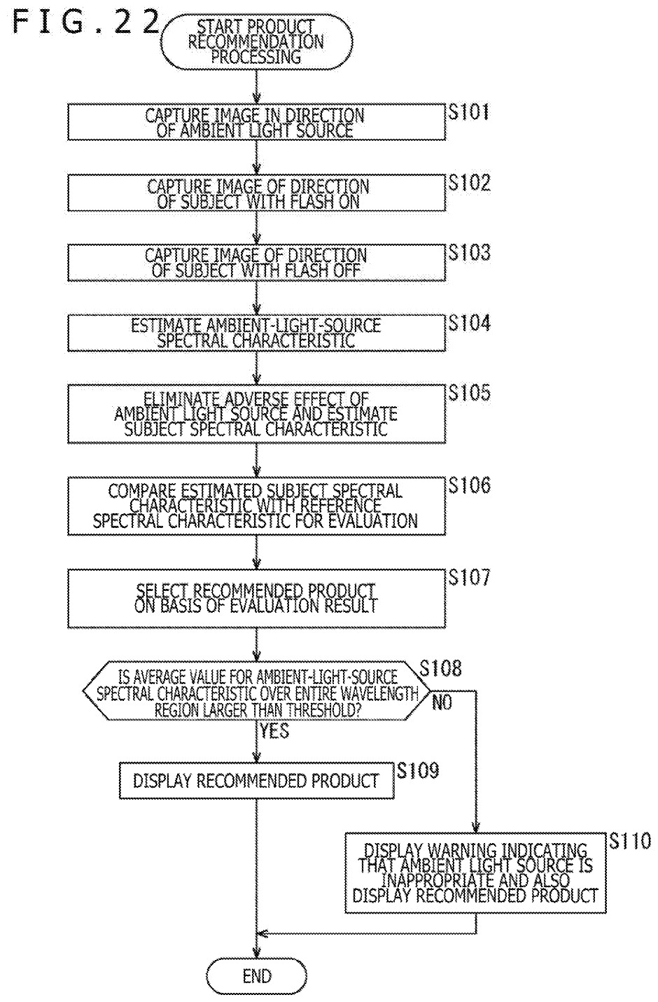

Now, with reference to a flowchart in FIG. 22, product recommendation processing by the recommended product presenting apparatus 11 in FIG. 21 will be described.

In step S101, when instructed to capture an image by the user operating the operation section 32, the ambient-light-source measuring section 121 captures an image of a direction in which a light source that may be used as an ambient light source is reliably present, and outputs the captured image to the ambient-light-source estimating section 122.

In step S102, the measurement section 31 controls the flash 31a to capture an image of the subject with the flash 31a on, and outputs the captured image to the subject spectral characteristic estimating section 34.

In step S103, the measurement section 31 captures an image of the subject with the flash 31a off, and outputs the captured image to the subject spectral characteristic estimating section 34.

In step S104, the ambient-light-source estimating section 122 estimates the spectral characteristic of the ambient light source from the images fed by the ambient-light-source measuring section 121, and outputs an estimation result to the subject spectral characteristic estimating section 34 and the inappropriate-light-source detecting section 41 as an ambient-light-source spectral characteristic.

In step S105, the subject spectral characteristic estimating section 34 uses the ambient-light-source spectral characteristic fed by the ambient-light-source estimating section 122 to eliminate the adverse effect of the ambient light source and estimates the spectral characteristic of the subject in the image from the images captured by the measurement section 31, and outputs an estimation result to the subject characteristic comparing section 35 as a subject spectral characteristic.

In step S106, the subject characteristic comparing section 35 compares the subject spectral characteristic corresponding to the estimation result with the reference spectral characteristic corresponding to the spectral characteristic of the preferred state stored in the characteristic classification storage section 36, and outputs a comparison result to the recommended product selecting section 37 as an evaluation result for evaluation of the skin of the user. At this time, the subject characteristic comparing section 35 outputs the subject spectral characteristic and the reference spectral characteristic of the preferred state to the recommended product selecting section 37 along with the evaluation result.

In step S107, the recommended product selecting section 37 selects (identifies) a product to be recommended from the products stored in the product storage section 38 as a recommended product (or recommended article), and outputs the recommended product to the output control section 39 on the basis of the evaluation result, the subject spectral characteristic, and the reference spectral characteristic of the preferred state.

In step S108, the inappropriate-light-source detecting section 41 determines whether the ambient light source is an inappropriate light source or not on the basis of whether or not the average intensity of the ambient-light-source spectral characteristic over the entire wavelength region fed by the ambient-light-source estimating section 122 is larger than a threshold corresponding to the average value of the subject spectral characteristic stored in the characteristic classification storage section 36 and the ambient light source is appropriate as an ambient light source with which the subject spectral characteristic is estimated, and outputs the determination result to the output control section 39.

In step S108, in a case where the average intensity of the ambient light source over the entire wavelength region is larger than the threshold corresponding to the average value of the subject spectral characteristic stored in the characteristic classification storage section 36 and where the ambient light source is determined to be appropriate as an ambient light source with which the subject spectral characteristic is estimated and not to be an inappropriate light source, the processing proceeds to step S109.

In step S109, the output control section 39 displays, on the display section 51 of the output section 40, product information related to the recommended product, and ends the processing.

On the other hand, in step S108, in a case where the average intensity of the ambient light source over the entire wavelength region is not larger than the threshold corresponding to the average value of the subject spectral characteristic stored in the characteristic classification storage section 36 and where the ambient light source is determined to be inappropriate as an ambient light source with which the subject spectral characteristic is estimated and to be an inappropriate light source, the processing proceeds to step S110.

In step S110, the output control section 39 displays, on the display section 51 of the output section 40, information indicating that the ambient light source is an inappropriate light source, along with the information related to the recommended product. Additionally, the output control section 39 vibrates the vibrator 52 to cause the speaker 53 to output a predetermined sound, thus indicating information that the ambient light source is an inappropriate light source and ending the processing.

The above-described processing enables the ambient-light-source spectral characteristic to be accurately estimated by separately providing a configuration for estimating the ambient-light-source spectral characteristic. Thus, the subject spectral characteristic can be estimated with the adverse effect of the ambient light source more appropriately eliminated.

Additionally, the ambient-light-source spectral characteristic estimated by the ambient-light-source measuring section 121 and the ambient-light-source estimating section 122 of the recommended product presenting apparatus 11 in FIG. 21 may be registered in, a cloud server or the like in association with an estimated location such that the ambient-light-source spectral characteristic can be utilized by, for example, the recommended product presenting apparatus 11 in FIG. 18, which includes neither of the ambient-light-source measuring section 121 and the ambient-light-source estimating section 122.

### <<6. Example of Execution by Using Software>>

Incidentally, the above-described series of steps of processing can be executed by using hardware but can also be executed by using software instead. In a case where the series of steps of processing are executed by using software, a program included in the software is installed from a recording medium into a computer integrated in dedicated hardware or, for example, a general-purpose computer in which various programs are installed such that the computer can execute various functions.

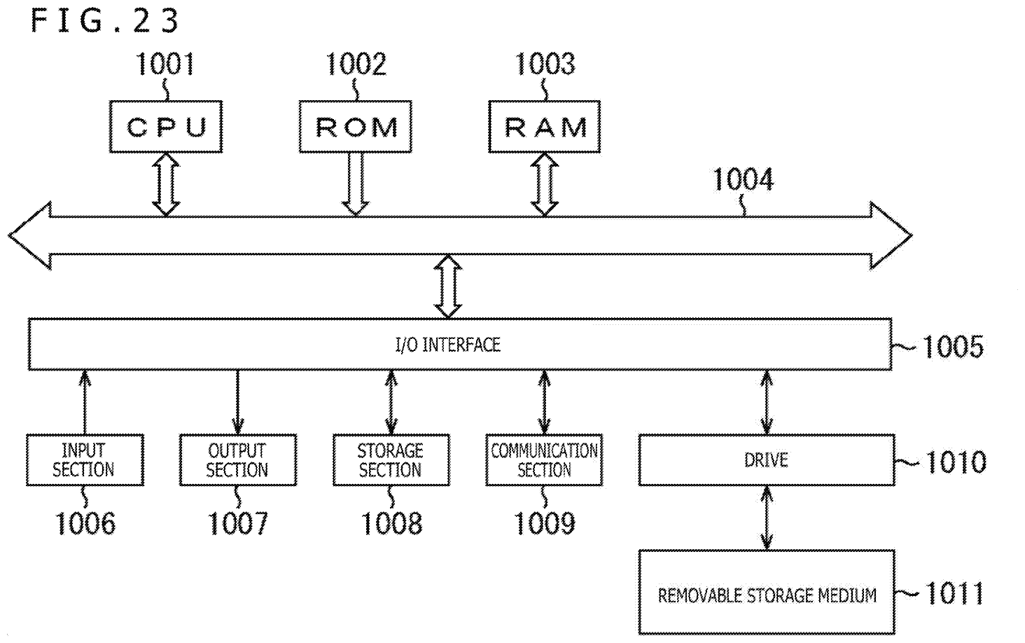

FIG. 23 illustrates a configuration example of a general-purpose computer. The personal computer includes a built-in CPU (Central Processing Unit) 1001. The CPU 1001 connects to an I/O interface 1005 via a bus 1004. The bus 1004 connects to a ROM (Read Only Memory) 1002 and a RAM (Random Access Memory) 1003.

The I/O interface 1005 connects to an input section 1006 including an input device such as a keyboard and a mouse through which the user inputs operation commands, an output section 1007 outputting processing operation screens and images of processing results to a display device, a storage section 1008 including a hard disk drive or the like in which programs and various data are stored, and a communication section 1009 including a LAN (Local Area Network) adapter or the like and executing communication processing via a network typified by the Internet. Additionally, the I/O interface 1005 connects to a drive 1010 reading and writing data from and to a removable storage medium 1011 such as a magnetic disk (including a flexible disk), an optical disc (including a CD-ROM (Compact Disc-Read Only Memory) or a DVD (Digital Versatile Disc)), a magneto-optical disc (including an MD (Mini Disc)), or a semiconductor memory.

The CPU 1001 executes various types of processing in accordance with programs stored in the ROM 1002 or programs read from the removable storage medium 1011 such as a magnetic disk, an optical disc, a magneto-optical disc, or a semiconductor memory and installed in the storage section 1008 and then loaded from the storage section 1008 into the RAM 1003. The RAM 1003 also appropriately stores data required for the CPU 1001 to execute various types of processing.

In the computer configured as described above, the CPU 1001 executes the above-described series of steps of processing by, for example, loading programs stored in the storage section 1008 into the RAM 1003 via the I/O interface 1005 and the bus 1004.

The programs executed by the computer (CPU 1001) can be provided by, for example, being recorded in the removable storage medium 1011 used as a package medium or the like. Additionally, the programs can be provided via a wired or wireless transmission medium such as a local area network, the Internet, or digital satellite broadcasting.

In the computer, the programs can be installed in the storage section 1008 via the I/O interface 1005 by installing the removable storage medium 1011 in the drive 1010. Additionally, the programs can be received by the communication section 1009 via a wired or wireless transmission medium and installed in the storage section 1008. The programs can otherwise be pre-installed in the ROM 1002 or the storage section 1008.

Note that the programs executed by the computer may be programs chronologically executing processing along the order described herein or executing processing in parallel or at required timings when, for example, the programs are invoked.

Note that the CPU 1001 in FIG. 23 implements the functions of the ambient-light-source estimating section 33, the subject spectral characteristic estimating section 34, the subject characteristic comparing section 35, the recommended product selecting section 37, the output control section 39, and the inappropriate-light-source detecting section 41 in FIG. 1, the subject characteristic classifying section 71 in FIG. 11, the measurement item selecting section 81 in FIG. 14, the ambient-light-source registering section 101 and the ambient-light-source selecting section 103 in FIG. 18, and the ambient-light-source estimating section 122 in FIG. 21. Additionally, the storage section 1008 in FIG. 23 implements the characteristic classification storage section 36 and the product storage section 38 in FIG. 1, FIG. 11, FIG. 14, FIG. 18, and FIG. 21, and the ambient-light-source storage section 102 in FIG. 18.

Additionally, the system as user herein means a set of a plurality of components (apparatuses, modules (parts), or the like) regardless of whether all the components are located inside the same housing. Accordingly, a plurality of apparatuses stored in different housings and connected together via a network and one apparatus including a plurality of modules stored in one housing are both systems.

Note that the embodiments of the present disclosure are not limited to the above-described embodiments but various changes may be made to the embodiments without departing from the spirits of the present disclosure.

For example, the present disclosure can be configured as cloud computing in which one function is shared and cooperatively processed by a plurality of apparatuses via a network.

Additionally, not only can the steps described above with reference to the flowchart be executed by a single apparatus but also be shared among a plurality of apparatuses.

Additionally, in a case where a plurality of processing tasks are included in one step, a plurality of the processing tasks in the one step can be executed in one apparatus or can be shared and executed by a plurality of apparatuses.

The present disclosure can be configured as described below.

<1> An image processing apparatus including:

an evaluation section evaluating a state of a subject in a captured image on the basis of a subject spectral characteristic corresponding to a spectral characteristic of the subject and a reference spectral characteristic.

<2> The image processing apparatus according to <1>, in which

the evaluation section evaluates the state of the subject on the basis of a difference between the subject spectral characteristic and the reference spectral characteristic.

<3> The image processing apparatus according to <2>, in which

the evaluation section outputs, as an evaluation result for the state of the subject, a comparison result of comparison of a difference square root sum between the subject spectral characteristic and the reference spectral characteristic with a predetermined threshold.

<4> The image processing apparatus according to <3>, in which

the predetermined threshold is a difference square root sum between an average value of subject spectral characteristics of a plurality of subjects and the reference spectral characteristic.

<5> The image processing apparatus according to any one of <1> to <4>, in which

the reference spectral characteristic is a spectral characteristic of a preferred state of the subject in the subject spectral characteristic.

<6> The image processing apparatus according to any one of <1> to <5>, further including:

an identification section identifying an article with a spectral characteristic applied to the subject to make the spectral characteristic of the subject more similar to the reference spectral characteristic, and

a display section displaying the article identified by the identification section.

<7> The image processing apparatus according to <6>, in which

in a case where, in an evaluation result for the state of the subject including a comparison result of comparison of a difference square root sum between the subject spectral characteristic and the reference spectral characteristic with the predetermined threshold, the difference square root sum between the subject spectral characteristic and the reference spectral characteristic is larger than the predetermined threshold,

the identification section identifies, on the basis of the difference between the subject spectral characteristic and the reference spectral characteristic, an article with a spectral characteristic applied to the subject to make the spectral characteristic of the subject more similar to the reference spectral characteristic.

<8> The image processing apparatus according to <6>, in which

the spectral characteristic applied to the subject to make the spectral characteristic of the subject more similar to the reference spectral characteristic is a spectral characteristic obtained by multiplying, for each wavelength, the difference between the subject spectral characteristic and the reference spectral characteristic by a predetermined coefficient and adding the subject spectral characteristic to a result of the multiplication.

<9> The image processing apparatus according to <6>, further including:

an article storage section storing information related to the article in association with information related to a spectral characteristic provided in the subject by application of the article, in which

the identification section identifies an article that is included in articles stored in the article storage section, the article involving a decrease in a difference square root sum between the spectral characteristic stored in association with the information and a spectral characteristic applied to the subject to make the spectral characteristic of the subject more similar to the reference spectral characteristic.

<10> The image processing apparatus according to <1>, in which

the evaluation section evaluates the state of the subject by classifying the subject spectral characteristic.

<11> The image processing apparatus according to <10>, in which

the evaluation section evaluates the state of the subject by dividing the subject spectral characteristic into a plurality of wavelength regions, classifying the subject spectral characteristic on the basis of a comparison result of comparison of a predetermined threshold with a difference square root sum for each wavelength resulting from the division, and obtaining a classification result as an evaluation index.

<12> The image processing apparatus according to <11>, further including:

an identification section identifying, on the basis of the evaluation index, an article with a spectral characteristic applied to the subject to make the spectral characteristic of the subject more similar to the reference spectral characteristic; and

a display section displaying the article identified by the identification section.

<13> The image processing apparatus according to <12>, further including:

an article storage section storing, for information related to the article, an index of the article with a spectral characteristic applied to the subject to make the spectral characteristic of the subject more similar to the reference spectral characteristic, in association with an evaluation index of the subject spectral characteristic, the index of the article being based on a difference between the subject spectral characteristic and the reference spectral characteristic, in which

the identification section identifies an article that is included in articles stored in the article storage section and that is stored in association with the evaluation index, as an article with a spectral characteristic applied to the subject to make the spectral characteristic of the subject more similar to the reference spectral characteristic.

<14> The image processing apparatus according to <13>, in which

the spectral characteristic applied to the subject to make the spectral characteristic of the subject more similar to the reference spectral characteristic is a spectral characteristic obtained by multiplying, for each wavelength, the difference between the subject spectral characteristic and the reference spectral characteristic by a predetermined coefficient and adding the subject spectral characteristic to a result of the multiplication.

<15> The image processing apparatus according to any one of <1> to <14>, further including:

an ambient-light-source spectral characteristic estimating section estimating a spectral characteristic of an ambient light source in a captured image as an ambient-light-source spectral characteristic, in which

the evaluation section evaluates a state of the subject in an image with an adverse effect of the ambient light source in the image reduced using the ambient-light-source spectral characteristic estimated from the image, on the basis of a difference between the reference spectral characteristic and the subject spectral characteristic of the subject.

<16> The image processing apparatus according to <15>, further including:

an ambient-light-source spectral characteristic storage section storing, in association with a measurement location, the ambient-light-source spectral characteristic estimated by the ambient-light-source spectral characteristic estimating section, in which

the evaluation section evaluates, on the basis of the subject spectral characteristic of the subject, the state of the subject in an image with the adverse effect of the ambient light source in the image reduced using an ambient-light-source spectral characteristic selected from ambient-light-source spectral characteristics stored in the ambient-light-source spectral characteristic storage section.

<17> The image processing apparatus according to <15>, further including:

an inappropriate-ambient-light-source detecting section detecting that the ambient light source is an inappropriate light source for the subject spectral characteristic on the basis of the ambient-light-source spectral characteristic estimated by the ambient-light-source spectral characteristic estimating section; and

a presentation section indicating that the ambient light source is an inappropriate light source in a case where the ambient light source is detected as an inappropriate light source.

<18> The image processing apparatus according to <17>, in which

the inappropriate-ambient-light-source detecting section detects that the light source is inappropriate on the basis of a comparison between an average value of the subject spectral characteristic in the ambient-light-source spectral characteristic estimated by the ambient-light-source spectral characteristic estimating section.

<19> An image processing method including:

evaluation processing of evaluating a state of a subject in a captured image on the basis of a reference spectral characteristic and a subject spectral characteristic corresponding to a spectral characteristic of the subject.

<20> A program causing an evaluation section evaluating a state of a subject in a captured image to function as a computer on the basis of a reference spectral characteristic and a subject spectral characteristic corresponding to a spectral characteristic of the subject.

### REFERENCE SIGNS LIST

11 Recommended product selecting apparatus
31 Recording section
31a Flash
32 Operation section
33 Ambient-light-source estimating section
34 Subject spectral characteristic estimating section
35 Subject characteristic comparing section
36 Characteristic classification storage section
37 Recommended product selecting section
38 Product storage section
39 Output control section
40 Output section
41 Inappropriate-light-source detecting section
51 Display section
52 Vibrator
53 Speaker
71 Subject characteristic classifying section
72 Characteristic classification storage section
73 Recommended product selecting section
74 Product storage section
81 Measurement item selecting section
101 Ambient-light-source registering section
102 Ambient-light-source storage section
103 Ambient-light-source selecting section
121 Ambient-light-source measuring section
122 Ambient-light-source estimating section

1. An image processing apparatus comprising:
an evaluation section evaluating a state of a subject in a captured image on a basis of a subject spectral characteristic corresponding to a spectral characteristic of the subject and a reference spectral characteristic.

2. The image processing apparatus according to claim 1, wherein
the evaluation section evaluates the state of the subject on a basis of a difference between the subject spectral characteristic and the reference spectral characteristic.

3. The image processing apparatus according to claim 2, wherein
the evaluation section outputs, as an evaluation result for the state of the subject, a comparison result of comparison of a difference square root sum between the subject spectral characteristic and the reference spectral characteristic with a predetermined threshold.

4. The image processing apparatus according to claim 3, wherein
the predetermined threshold is a difference square root sum between an average value of subject spectral characteristics of a plurality of subjects and the reference spectral characteristic.

5. The image processing apparatus according to claim 1, wherein
the reference spectral characteristic is a spectral characteristic of a preferred state of the subject in the subject spectral characteristic.

6. The image processing apparatus according to claim 1, further comprising:
an identification section identifying an article with a spectral characteristic applied to the subject to make the spectral characteristic of the subject more similar to the reference spectral characteristic, and
a display section displaying the article identified by the identification section.

7. The image processing apparatus according to claim 6, wherein
in a case where, in an evaluation result for the state of the subject including a comparison result of comparison of a difference square root sum between the subject spectral characteristic and the reference spectral characteristic with the predetermined threshold, the difference square root sum between the subject spectral characteristic and the reference spectral characteristic is larger than the predetermined threshold,
the identification section identifies, on a basis of the difference between the subject spectral characteristic and the reference spectral characteristic, an article with a spectral characteristic applied to the subject to make the spectral characteristic of the subject more similar to the reference spectral characteristic.

8. The image processing apparatus according to claim 6, wherein
the spectral characteristic applied to the subject to make the spectral characteristic of the subject more similar to the reference spectral characteristic is a spectral characteristic obtained by multiplying, for each wavelength, the difference between the subject spectral characteristic and the reference spectral characteristic by a predetermined coefficient and adding the subject spectral characteristic to a result of the multiplication.

9. The image processing apparatus according to claim 6, further comprising:
an article storage section storing information related to the article in association with information related to a spectral characteristic provided in the subject by application of the article, wherein
the identification section identifies an article that is included in articles stored in the article storage section, the article involving a decrease in a difference square root sum between the spectral characteristic stored in association with the information and a spectral characteristic applied to the subject to make the spectral characteristic of the subject more similar to the reference spectral characteristic.

10. The image processing apparatus according to claim 1, wherein
the evaluation section evaluates the state of the subject by classifying the subject spectral characteristic.

11. The image processing apparatus according to claim 10, wherein
the evaluation section evaluates the state of the subject by dividing the subject spectral characteristic into a plurality of wavelength regions, classifying the subject spectral characteristic on a basis of a comparison result of comparison of a predetermined threshold with a difference square root sum for each wavelength region resulting from the division, and obtaining a classification result as an evaluation index.

12. The image processing apparatus according to claim 11, further comprising:
an identification section identifying, on a basis of the evaluation index, an article with a spectral characteristic applied to the subject to make the spectral characteristic of the subject more similar to the reference spectral characteristic; and
a display section displaying the article identified by the identification section.

13. The image processing apparatus according to claim 12, further comprising:
an article storage section storing, for information related to the article, an index of the article with a spectral characteristic applied to the subject to make the spectral characteristic of the subject more similar to the reference spectral characteristic, in association with an evaluation index of the subject spectral characteristic, the index of the article being based on a difference between the subject spectral characteristic and the reference spectral characteristic, wherein
the identification section identifies an article that is included in articles stored in the article storage section and that is stored in association with the evaluation index, as an article with a spectral characteristic applied to the subject to make the spectral characteristic of the subject more similar to the reference spectral characteristic.

14. The image processing apparatus according to claim 13, wherein
the spectral characteristic applied to the subject to make the spectral characteristic of the subject more similar to the reference spectral characteristic is a spectral characteristic obtained by multiplying, for each wavelength, the difference between the subject spectral characteristic and the reference spectral characteristic by a predetermined coefficient and adding the subject spectral characteristic to a result of the multiplication.

15. The image processing apparatus according to claim 1, further comprising:
an ambient-light-source spectral characteristic estimating section estimating a spectral characteristic of an ambient light source in a captured image as an ambient-light-source spectral characteristic, wherein
the evaluation section evaluates a state of the subject in an image with an adverse effect of the ambient light source in the image reduced using the ambient-light-source spectral characteristic estimated from the image, on a basis of a difference between the reference spectral characteristic and the subject spectral characteristic of the subject.

16. The image processing apparatus according to claim 15, further comprising:
an ambient-light-source spectral characteristic storage section storing, in association with a measurement location, the ambient-light-source spectral characteristic estimated by the ambient-light-source spectral characteristic estimating section, wherein
the evaluation section evaluates, on a basis of the subject spectral characteristic of the subject, the state of the subject in an image with the adverse effect of the ambient light source in the image reduced using an ambient-light-source spectral characteristic selected from ambient-light-source spectral characteristics stored in the ambient-light-source spectral characteristic storage section.

17. The image processing apparatus according to claim 15, further comprising:
an inappropriate-ambient-light-source detecting section detecting that the ambient light source is an inappropriate light source for the subject spectral characteristic on a basis of the ambient-light-source spectral characteristic estimated by the ambient-light-source spectral characteristic estimating section; and
a presentation section indicating that the ambient light source is an inappropriate light source in a case where the ambient light source is detected as an inappropriate light source.

18. The image processing apparatus according to claim 17, wherein
the inappropriate-ambient-light-source detecting section detects that the light source is inappropriate on a basis of a comparison between an average value of the subject spectral characteristic in the ambient-light-source spectral characteristic estimated by the ambient-light-source spectral characteristic estimating section.

19. An image processing method comprising:
evaluation processing of evaluating a state of a subject in a captured image on a basis of a reference spectral characteristic and a subject spectral characteristic corresponding to a spectral characteristic of the subject.

20. A program causing an evaluation section evaluating a state of a subject in a captured image to function as a computer on a basis of a reference spectral characteristic and a subject spectral characteristic corresponding to a spectral characteristic of the subject.
